(19) United States
(12) Patent Application Publication
(10) Pub. No.: US 2016/0319044 A1
(43) Pub. Date: Nov. 3, 2016

(54) 13C LABELED STARCH/ALPHA LIMITED DEXTRINS DIGESTION BREATH TEST

(71) Applicant: Baylor College of Medicine, Houston, TX (US)

(72) Inventors: Antone Robert Opekun, JR., Houston, TX (US); Buford L. Nichols, JR., Houston, TX (US); Mark A. Gilger, Houston, TX (US)

(21) Appl. No.: 15/083,048

(22) Filed: Mar. 28, 2016

Related U.S. Application Data
(60) Provisional application No. 62/139,260, filed on Mar. 27, 2015.

(51) Int. Cl. C08B 30/18; G01N 33/497

(52) U.S. Cl. CPC C08B 30/18; G01N 33/497

(57) ABSTRACT

The present invention is directed to a composition and method for detecting intestinal deficiency. In particular, a ¹³C-α-limit dextrin that is specifically hydrolyzed by brush border intestinal enzymes is disclosed herein. The ¹³C-α-limit dextrin as disclosed herein may be used as a substrate in a bi-phasic breath test to detect intestinal glucoamylase deficiency and/or abnormal brush border intestinal enzyme activity.

This application claims priority to U.S. Provisional Application No. 62/139,260, filed Mar. 27, 2015, which is incorporated by reference herein in its entirety.

### STATEMENT REGARDING FEDERALLY SPONSORED RESEARCH OR DEVELOPMENT

This invention was made with government support under NIDDK P30 Grant DK56338 awarded by United States Department of Health and Human Services National Institutes of Health. The government has certain rights in the invention.

### TECHNICAL FIELD

The field of subject matter of the invention includes at least molecular biology, biochemistry, chemistry, nutrition and medicine. In specific aspects, the field of subject matter of the invention includes functional gastrointestinal disease in adults and children, starch maldigestion, management of childhood gastrointestinal and other genetic, infectious and metabolic diseases.

### BACKGROUND OF THE INVENTION

Abdominal pain (AP)-related functional gastrointestinal disorders (FGID) and digestive enzyme deficiency syndromes are characterized by chronic recurrent abdominal pain without evidence of an inflammatory, anatomic or neoplastic etiology. Abdominal pain-related functional gastrointestinal disorders are defined by chronic continuous or intermittent abdominal discomfort that occurs at a minimum of once a week over a two-month period and often associated with inconvenient changes in fecal excretion pattern and character, such as diarrhea or constipation (Rasquin-Weber et al., 1999). From six to eighteen percent of middle school and high school-aged children suffer from these disorders (Chitkara et al., 2005). Abdominal pain-related functional gastrointestinal disorders are a frequent cause of health care visits and invasive medical procedures. Also, these disorders significantly impact activities of daily living and decrease the quality of life in those affected (Youssef et al., 2006). Although these disorders do not appear to have associated mortality, they do place a significant burden on society such as direct and indirect costs to the family and work place due to competing parental responsibilities and significant health care costs to the system at large (Lane et al., 2009). Similarly, enzyme deficiency syndromes, such as congenital disaccharidase deficiency, celiac disease, inflammatory bowel disease, primary mucosal atrophy, drug or toxin-induced secondary mucosal injury, irritable or functional bowel, gastroparesis, and parasitic infections are known to cause similar clinical manifestations and may have significant adverse outcomes. As such, the etiology should be ascertained and effective treatment identified.

Functional gastrointestinal disorders have been categorized into sub-types based on associated symptoms and relationship to defecation (functional dyspepsia (FD), irritable bowel syndrome (IBS) and functional abdominal pain (FAP)) (Rasquin et al., 2006). Functional dyspepsia indicates that organic etiologies, including esophagitis, gastritis, duodenitis and ulcers, were excluded and suggests that the discomfort is related to elusive physiological small intestinal phenomena distal to the pylorus. An IBS subtype has been described as similar “abdominal discomfort” associated with at least two of the following additional intermittent features: improvement with defecation, change in frequency of stool and change in stool form. With functional abdominal pain (FAP) in children, the relationship between discomfort and change in stool character is vague or absent. In daily practice, the distinction between FAP and IBS is sometimes not clear and patients may change from one category to another over time (Walker et al., 1998 and Chitkara et al., 2009). Lactase and sucrase insufficiency have been well described. Both lactase and sucrose insufficiency have similar IBS-like symptoms and respond to treatment (Lomer et al., 2008 and Treem et al., 1999). Although lactase and sucrase insufficiency exclude a “functional” diagnosis, the premise is that they model other glucosidase insufficiency conditions including isomaltase and maltase-glucoamylase deficiency, which have been largely ignored due to the absence of testing methods.

Though numerous etiologies have been proposed as causes for AP-FGID, faulty biochemical digestive processes are believed to be major contributors (Moukarzel et al., 2002; Fernandez-Banares et al., 1993; Goldstein et al., 2000; and Nordgaard and Mortensen, 1995). The enigmatic IBS symptom constellation of bloating, abdominal pain, nausea, mucus, diarrhea and constipation are reminiscent of the para-syndrome often seen with lactose intolerance. These features are also the side effects observed with use of the anti-diabetic drug acarbose, a potent α-glucosidase inhibitor that causes carbohydrate malabsorption (Ladas et al., 1992) and toxic attributes associated with cancer chemotherapy.

Starches are the main source of calories in a Western child's diet (60-90%) (Nicklas et al., 1996), and, after partial and incomplete digestion by pancreatic amylase, complete starch digestion is ultimately dependent upon effective luminal, mucosal-bound α-glucosidase activity, including the 4 domains of MGAM, maltase glucamylase, and SI, sucrose isomaltase. Recently, it has been demonstrated that mucosal MGAM plays a role in end-stage or end-tier starch digestion and prandial glucose homeostasis of mice (Nichols et al., 2009). Children with impaired mucosal α-glucosidases have remarkably similar features to IBS (Treem et al., 1999). The proximal maldigestion results in the appearance of fermentable oligosaccharides in the distal colon (Lee et al., 2004). The end-products of fermentation from malabsorbed oligosaccharides facilitate excessive lower GI gas production and the production of bioactive metabolites, and, in severe cases, causes chronic diarrhea (Jenkins et al., 1981). The idea that mucosal/brush border α-glucosidase activity-loss could be etiologically involved in FGID, IBS, and various congenital and acquired conditions has not yet been thoroughly considered (Lebenthal et al., 1994; Simadibrata et al., 2003; Halmos et al., 2014).

Luminal surface (brush border) mucosal enzyme deficiencies leads to symptoms. Congenital sucrase-isomaltase deficiency (CSID) is an infrequent, but sometimes a severe problem for afflicted individuals with many of the same clinical FGID features (Karnsakul et al., 2002). Most children with CSID also have poor starch digestion, and there also appears to be a spectrum of inadequacy of mucosal starch digestive ability in children with symptoms that do not exhibit the severity of those with specific mutations (Auricchio et al., 1971). This appears related to the numerous mutations that have been describe since first reports appeared (Alfalah et al., 2009). Inflammatory bowel disease and viral-induced injury are examples of acquired mucosal enzyme deficiencies. (Mehta and Blecker; 1998)

Food carbohydrates must be digested to soluble monosaccharides in the small intestine. The final stage of this process is accomplished by a family of membrane bound luminal enzymes, called disaccharidases, before absorption will occur (Jenkins et al., 1981). Invasive, clinical disaccharidase assays were developed by Dahlqvist in the 1960's (Dahlqvist et al., 1963 and Borgstrom et al., 1957). The best-studied disaccharidases are lactase (Enzyme Commission (EC) 3.2.1.62-3.2.1.10) and sucrase-isomaltase (EC 3.2.1.48-3.2.1.10). Lactase hydrolysis milk-sugar, lactose, to glucose and galactose to facilitate receptor mediated absorption and sucrase-isomaltase undergoes trypsin-mediated cleavage to provide two separate active forms that hydrolyze table sugar, sucrose, and hydrolyzes alpha bonds of soluble oligosaccharides to produce shorter-chain glucose oligomers and free glucose, respectively, Mucosal maltase-glucoamylase (MGAM, EC 3.2.1.20 and EC 3.2.1.3) works in concert with isomaltase to complete starch hydrolysis to glucose. Because of recognizable clinical symptoms, CSID has been prototypically studied at clinical, proteomic and genomic levels (Robayo-Torres et al., 2006), but less attention has been given to starch maldigestion due to its complexity. Sucrose and starch maldigestion have been associated with the various irritable bowel syndromes and respond to dietary restrictions (Opekun et al., 2014; Gibson et al., 2015; http://www.gastrojournal.org/article/S0016-5085(14)60780-0/pdf; http://www.ncbi.nim.nih.gov/pubmed/25680668).

The role of salivary and pancreatic α-amylase activities in whole starch digestion are well known in the commercial production of α-limit dextrins and oligosaccharides in vitro, but the roles of mucosal-bound α-glucosidase activities are less understood.

Alpha-amylase releases only 4% of the glucose present in amylopectin, and the remaining 96% of products are soluble glucose oligomers with a variable pattern of lengths that must be hydrolyzed at the apical membrane surface by mucosal-generated α-glucosidases before absorption and transport can occur (Wright, 2006). Oligomers, including maltose (G2) and lengths through G40 dextrins, require further digestion. Mucosal α-glucosidases release free glucose from the non-reducing ends of the soluble oligomers to permit enterocyte absorption, otherwise malabsorption and excessive colonic fermentation occurs that results in symptoms (Lee et al., 2004). Undigested soluble starch is vulnerable to colonic fermentation that results in production of short-chain fatty acids and acidification that affects colonic mucosal cytotoxicity (Van Munster et al., 1994) and results in adverse symptoms.

Differentiation of α-amylase from α-glucosidase activity was first reported in 1880 after discovery of maltase activity in the small intestine and its absence in the pancreas (Brown and Heron, 1880). In the 1960's Dahlqvist and others (Dahlqvist et al., 1963; Bayless and Christopher, 1969) found that four different maltase activities could be identified in the human jejunum; two were isolated with SI and two, that were free of other disaccharidase activities, were called MGAM. In the 1980's Heymann et al. (Heymann et al., 1995), proved that mucosal maltase activities are a subgroup of the α-glucosidases, and that while substrate specificities of SI and MGAM overlap, the activity of MGAM was approximately 100-fold greater, but concentration of SI was 20-fold greater. MGAM activities appear responsible for digestion at low starch intakes, but are inhibited by large intakes when slower SI activity constrains the digestion. The presence of overlapping activities appears to have evolved to accommodate seasonal diet variances and possibly ensure survival. Optimal starch digestion is critical to glycemic control (Jenkins et al., 1987), influences risk of obesity (Slavin, 2003) and appears to influence gut immune functions and cancer risks (Bird et al., 2000). Mammalian maltase and sucrase activities appear to be carefully regulated at the enterocyte transcriptional level, and expression can be regulated by glucocorticoids (Galand, 1989). The simultaneous regulation of the concentration of SI and MGAM in the microvillus membranes suggests that they may be subjected to related control mechanisms and aberration of expression may occur.

The etiology of acquired disaccharidase insufficiency is incomplete, but it has been associated with viral enterocyte injury (Taylor et al., 1971), with rotavirus and severe phenotypes of malnutrition being typical examples. The clinical and nutritional implications of shared substrate α-glucosidase activities by MGAM and SI suggest that both enzyme complexes, sharing four conserved catalytic sites for mucosal maltose and starch oligomer digestion, are important for ideal digestion (Aurrichio, 1971). Furthermore, shared amino acid homology (˜59%) among MGAM and SI suggest that MGAM may have evolved as a secondary survival pathway for starch digestion (Nichols et al., 1998).

Amyloglucosidase (AMG) is a common food supplement. AMG is a potent inverting exo-acting hydrolase-releasing glucose from the non-reducing ends of oligosaccharides which is used as a component of nutritional supplements and in the food industry to modify dietary starches (Sauer et al., 2000). AMG is distinguished by releasing alpha-glucose from the common substrates having alpha-glucosidic linkages (Guzman-Maldonado and Paredes-Lopez, 1995) and is used in the final step of food analysis to determine soluble dietary fiber content from insoluble content (Goncalvez et al., 1998). It is derived from non-pathogenic strains of Aspergillus species and Saccharomycopsis and, like MGAM, is inhibited by acarbose (Natarajan and Sierks, 1996). The majority of α-glucoamylases are multi-domain enzymes consisting of a catalytic domain connected to a starch-binding domain by an O-glycosylated linker region. The free enzyme is active in aqueous media and lipid vesicles (Li et al., 2007). Three-dimensional structures have been determined for free and inhibitor-complexed glucoamylases. The catalytic domain folds are a twisted (alpha/alpha)(6)-barrel with a central funnel-shaped active site, while the starch-binding domain folds as an antiparallel beta-barrel and has two binding sites for starch (Sauer et al., 2000). The functional role of a loop region is highly conserved among α-glucoamylases (Natarajan and Sierks, 1996). As such, it appears to be a model to illustrate in vivo measured changes of digestion or relative physiological states.

Kinniburgh (U.S. Patent Publication 2006/0263296) discloses a method and a kit for use in the performance of a ¹³CO₂ breath test of glucose metabolism. In Kinniburgh, the subject gives an initial unlabeled breath sample and a second breath sample comprising labeled and unlabeled sugar. The first breath sample is used to establish a baseline ¹³CO₂/¹²CO₂ ratio. In some examples, the method of Kinniburgh calls for an unlabeled sugar solution to be administered to the subject which is followed by additional breath sampling. Before the second breath sample, a subject is given a labeled sugar/unlabeled sugar solution. The second breath sample is collected and the difference in the ¹³CO₂/¹²CO₂ ratio before and after the administration of the labeled sugar is calculated. Kinniburgh asserts that this method is capable of distinguishing the degree to which a patient's intrinsic glucose (post-absorptive) metabolic capacity is compromised, but no mention is given to its application as a comparative, reference substrate to evaluate the efficiency of pre-absorptive hydrolysis (digestion) of disaccharides and oligosaccharides, a critical consideration for normalization of inter-subject representative of complex carbohydrate digestion and interpretation of test results with respect to age-dependent habitus. However, Kinniburgh's consideration only applies to the need to adjust intra-subject metabolic differences without consideration of any specific application or circumstance.

In this present disclosure, a need exists to provide a breath test that can be used to determine whether a specific enzyme is responsible for a patient's carbohydrate (starch and oligosaccharide) digestive capacity.

### BRIEF SUMMARY OF THE INVENTION

The present invention is directed to a composition and method for detecting intestinal disaccharidase deficiency. In particular, the present disclosure presents ¹³C-α-limit dextrins that are capable of being hydrolyzed by specific brush border intestinal enzymes, in isolation of pancreatic amylase, and used in the method for detecting intestinal mucosal isomaltase and glucoamylase deficiency. The ¹³C-α-limit dextrins as disclosed herein may be used as an ingested substrate in a breath test to detect intestinal isomaltase and glucoamylase deficiency and/or abnormal brush border intestinal enzyme activity, beyond the natural limits of pancreatic α-amylase. ¹³C-α-limit dextrins are derived from the partial hydrolysis of highly complex ¹³C-labelled starches produced by plants or algae that were commercially grown in a highly enriched atmosphere for the purpose of harvesting substrate for refinement.

An embodiment of the present invention includes a ¹³C-α-limit dextrin composition having the general formula:

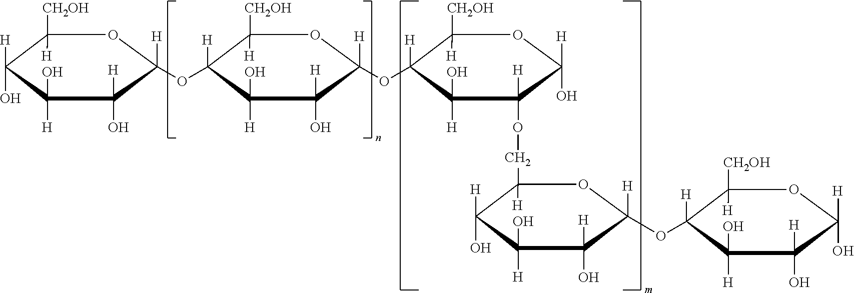

In some embodiments, the ¹³C-α-limit dextrin composition is uniformly enriched with ¹³C or ¹⁴C. In general, the composition has between 5 and 60 glucose units, n is between 3 and 58, m is between 0 and 10, and/or the sum of m and n is between 3 and 58. In specific embodiments where m is 1 or more, the ratio of m and n is between 1 to 58 and 1 to 3. In some embodiments, n is between 11 and 30. In other embodiments, n is between 15 and 26. In further embodiments, n is between 19 and 22. In some aspects, m is between 1 and 5. In further aspects, m is between 1 and 3. In specific aspects, m is 1 or 2. In one embodiment, the sum of m and n is 20. In some embodiments, the composition comprises a reference super-dose substrate of ¹³C-glucose. In further embodiments, the composition is a component of a solution. In certain embodiments, the ¹³C-α-limit dextrin composition is included in a 100 mL solution containing 50 milligrams of ¹³C-α-limit dextrins and 20 grams of mixed glucose polymers derived from corn starch (Polycose®, Abbott Laboratories, Columbus Ohio) that serves as a loading meal (80 kcal). In some embodiments, the ¹³C-α-limit dextrin composition is included in a capsule for administering to a subject. In further embodiments, the capsule includes 50 milligrams of ¹³C-α-limit dextrin. In some embodiments, the ¹³C-α-limit dextrin composition is administered to a subject with a separate load meal. In further embodiments, the composition is included in a kit. In some embodiments, a kit incorporates a reference super-dose substrate of ¹³C-glucose. In some embodiments, a reference super-dose is three to six times greater than the primary target substrate.

In one aspect of the invention, a method for detecting intestinal enzyme deficiency in a subject comprises the steps of administering to a subject a uniformly ¹³C or ¹⁴C labeled glucose substrate, wherein the uniformly ¹³C or ¹⁴C glucose substrate is metabolized to produce ¹³CO₂ or ¹⁴CO₂, measuring the change in ¹³CO₂ or ¹⁴CO₂ enrichment resulting from the metabolized uniformly ¹³C or ¹⁴C labeled glucose substrate by collecting breath samples from the subject, administering to the subject a uniformly ¹³C or ¹⁴C labeled substrate, wherein the uniformly ¹³C or ¹⁴C labeled substrate is metabolized to produce ¹³CO₂ or ¹⁴CO₂, measuring the ¹³CO₂ or ¹⁴CO₂ resulting from the metabolized uniformly ¹³C or ¹⁴C labeled substrate by collecting breath samples from the subject, and comparing the measured ¹³CO₂ or ¹⁴CO₂ resulting from the metabolized uniformly ¹³C or ¹⁴C labeled substrate with the measured ¹³CO₂ or ¹⁴CO₂ resulting from the metabolized uniformly ¹³C or ¹⁴C labeled glucose. In some embodiments, the intestinal enzyme deficiency may result from a genetic mutation on at least one of the sucrose-isomaltase and maltase-glucoamylase genes. In some embodiments, the intestinal enzyme deficiency is an acquired intestinal enzyme deficiency.

An additional embodiment of the present invention includes a method for detecting intestinal enzyme deficiency in a subject comprising the steps of: administering to the subject a uniformly ¹³C or ¹⁴C isotopically-labeled substrate, wherein the uniformly ¹³C or ¹⁴C isotopically-labeled substrate is metabolized to produce ¹³CO₂ or ¹⁴CO₂, measuring the timed change in ¹³CO₂ or ¹⁴CO₂ enrichment resulting from the metabolized uniformly ¹³C or ¹⁴C isotopically-labeled substrate, such as ¹⁴C-LDx, ¹³C-LDx, ¹⁴C-sucrose, or ¹³C-sucrose, by collecting breath samples from the subject, followed by administration to a subject a uniformly ¹³C or ¹⁴C isotopically-labeled glucose to adjust for variations in individual habitus and metabolism, wherein the uniformly ¹³C or ¹⁴C glucose is metabolized to produce ¹³CO₂ or ¹⁴CO₂, measuring the rate of the ¹³CO₂ or ¹⁴CO₂ production resulting from metabolism of uniformly ¹³C or ¹⁴C isotopically-labeled glucose by collecting breath samples from the subject, comparing the measured ¹³CO₂ or CO₂ resulting from the metabolism of uniformly ¹³C or ¹⁴C isotopically-labeled substrate with the measured ¹³CO₂ or ¹⁴CO₂ resulting from the metabolism of uniformly ¹³C or ¹⁴C isotopically-labeled glucose. In some embodiments, the method further comprises making a mathematical comparison (ratio) between the resultant values. In some embodiments, the breath samples are timed breath samples. In some embodiments, a proportional adjustment for superdosing of labeled glucose is performed prior to measuring ¹³CO₂ or ¹⁴CO₂. In particular embodiments, the test substrates are ¹³C-α-limit dextrins or ¹³C starches that are capable of being specifically hydrolyzed by proximal brush border-bound intestinal enzymes. In some embodiments, the test substrate is ¹⁴C-sucrose or ¹³C-sucrose. In specific embodiments, the test substrate is a ¹³C isotopically-labeled LDx substrate, the comparative reference substrate is ¹³C isotopically-labeled glucose and the test principle is illustrated below when eleven healthy, asymptomatic subjects underwent serial ¹³C isotopically-labeled LDx substrate and ¹³C isotopically-labeled glucose breath testing, over two separate days and from these data, the best representative sampling time-points were determined to be forty-five minutes, sixty minutes and seventy-five minutes and these data established a lower-limit diagnostic cut-off value for the ¹³C-LDx coefficient of glucose oxidation to be 70 percent based upon the margin of errors observed among the resultant mean oxidation data values for the forty-five minute, sixty minute and seventy-five minute time-points. In some embodiments, the intestinal enzyme deficiency may result from a genetic mutation on at least one of the sucrose-isomaltase and maltase-glucoamylase genes. In some embodiments, the intestinal enzyme deficiency is an acquired intestinal enzyme deficiency.

In one embodiment, a method for detecting intestinal enzyme deficiency in a subject comprises the steps of: collecting a breath sample for measurement of baseline (natural enrichment)¹³C or ¹⁴C labeled carbon dioxide for subsequent, timed comparisons (change in enrichments), administering to a subject a uniformly ¹³C or ¹⁴C labeled α-limit dextrin substrate (or other target substrate) with concomitant excipient load of unlabeled dextrins (or unlabeled other target substrate), wherein the uniformly ¹³C or ¹⁴C labeled α-limit dextrin substrate (or other target substrate) is metabolized to produce ¹³CO₂ or ¹⁴CO₂, measuring the timed change in ¹³CO₂ or ¹⁴CO₂ enrichment resulting from the metabolized uniformly ¹³C or ¹⁴C labeled α-limit dextrin substrate (or other target substrate) by collecting breath samples from the subject, administering to the subject a uniformly ¹³C or ¹⁴C labeled glucose substrate, wherein the uniformly ¹³C or ¹⁴C labeled glucose substrate is metabolized to produce ¹³CO₂ or ¹⁴CO₂, measuring the timed change of ¹³CO₂ or ¹⁴CO₂ enrichment resulting from the metabolized uniformly ¹³C or ¹⁴C labeled glucose substrate by collecting breath samples from the subject, and comparing the measured ¹³CO₂ or ¹⁴CO₂ resulting from the metabolized uniformly ¹³C or ¹⁴C labeled α-limit dextrin substrate (or other target substrate) with the measured ¹³CO₂ or ¹⁴CO₂ resulting from the metabolized uniformly ¹³C or ¹⁴C labeled glucose after a proportional adjustment for superdosing of uniformly ¹³C or ¹⁴C labeled glucose substrate and expressing the resultant values as a ratio. In some embodiments, the other target substrate is ¹³C or ¹⁴C-labeled sucrose. In some embodiments, the intestinal enzyme deficiency may result from a genetic mutation on at least one of the sucrose-isomaltase and maltase-glucoamylase genes. In some embodiments, the intestinal enzyme deficiency is an acquired intestinal enzyme deficiency.

A further embodiment of the present invention includes a method for monitoring a subject having been diagnosed with intestinal enzyme deficiency comprising the steps of: administering to the subject a uniformly ¹³C or ¹⁴C isotopically-labeled substrate, wherein the uniformly ¹³C or ¹⁴C isotopically-labeled substrate is metabolized to produce ¹³CO₂ or ¹⁴CO₂, measuring the timed change in ¹³CO₂ or ¹⁴CO₂ enrichment resulting from the metabolized uniformly ¹³C or ¹⁴C isotopically-labeled substrate, such as ¹³C-LDx, by collecting breath samples from the subject, followed by administration to a subject a uniformly ¹³C or ¹⁴C isotopically-labeled glucose to adjust for variations in individual habitus and metabolism, wherein the uniformly ¹³C or ¹⁴C glucose is metabolized to produce ¹³CO₂ or ¹⁴CO₂, measuring the rate of the ¹³CO₂ or ¹⁴CO₂ production resulting from metabolism of uniformly ¹³C or ¹⁴C isotopically-labeled glucose by collecting breath samples from the subject, comparing the measured ¹³CO₂ or ¹⁴CO₂ resulting from the metabolism of uniformly ¹³C or ¹⁴C isotopically-labeled substrate with the measured ¹³CO₂ or CO₂ resulting from the metabolism of uniformly ¹³C or ¹⁴C isotopically-labeled glucose. In some embodiments, the method further comprises making a mathematical comparison (ratio) between the resultant values. In some embodiments, the breath samples are timed breath samples. In some embodiments, a proportional adjustment for superdosing of labeled glucose is performed prior to measuring ¹³CO₂ or ¹⁴CO₂. In some embodiments, the uniformly ¹³C or ¹⁴C isotopically-labeled substrate is ¹³C-sucrose or ¹⁴C-sucrose. In some embodiments, the intestinal enzyme deficiency may result from a genetic mutation on at least one of the sucrose-isomaltase and maltase-glucoamylase genes. In some embodiments, the intestinal enzyme deficiency is an acquired intestinal enzyme deficiency.

Some embodiments of the invention are directed towards a method for treating a subject having been diagnosed with intestinal enzyme deficiency comprising the steps of administering to the subject a uniformly ¹³C or ¹⁴C isotopically-labeled substrate, wherein the uniformly ¹³C or ¹⁴C isotopically-labeled substrate is metabolized to produce ¹³CO₂ or ¹⁴CO₂, measuring the timed change in ¹³CO₂ or ¹⁴CO₂ enrichment resulting from the metabolized uniformly ¹³C or ¹⁴C isotopically-labeled substrate, such as ¹³C-LDx, by collecting breath samples from the subject, followed by administration to a subject a uniformly ¹³C or ¹⁴C isotopically-labeled glucose to adjust for variations in individual habitus and metabolism, wherein the uniformly ¹³C or ¹⁴C glucose is metabolized to produce ¹³CO₂ or ¹⁴CO₂, measuring the rate of the ¹³CO₂ or ¹⁴CO₂ production resulting from metabolism of uniformly ¹³C or ¹⁴C isotopically-labeled glucose by collecting breath samples from the subject, comparing the measured ¹³CO₂ or ¹⁴CO₂ resulting from the metabolism of uniformly ¹³C or ¹⁴C isotopically-labeled substrate with the measured ¹³CO₂ or ¹⁴CO₂ resulting from the metabolism of uniformly ¹³C or ¹⁴C isotopically-labeled glucose. In some aspects, a method for treating a subject having been diagnosed with intestinal enzyme deficiency comprises the step of administering to the subject amyloglucosidase. In some embodiments, the method further comprises making a mathematical comparison (ratio) between the resultant values. In some embodiments, the breath samples are timed breath samples. In some embodiments, a proportional adjustment for superdosing of labeled glucose is performed prior to measuring ¹³CO₂ or ¹⁴CO₂. In some embodiments, the intestinal enzyme deficiency may result from a genetic mutation on at least one of the sucrose-isomaltase and maltase-glucoamylase genes. In some embodiments, the intestinal enzyme deficiency is an acquired intestinal enzyme deficiency.

In additional embodiments, the method described herein is used for monitoring a subject having been diagnosed with congenital or acquired intestinal enzyme deficiency. In other embodiments, the method described herein is used for evaluating the effects of food additives for adverse effects on intestinal enzyme activity and impaired digestion of carbohydrates and particularly starches, oligosaccharides and disaccharides. In some embodiments, intestinal enzyme deficiency is a deficiency in a glycoside hydrolase. In some embodiments, intestinal enzyme deficiency is a deficiency in a disaccharidase. In some embodiments, intestinal enzyme deficiency includes deficiency of isomaltase, glycoamylase, lactase, sucrose, amyloglucosidase, and α-glucosidase. In some embodiments an enzyme deficiency may result from low enzyme activity, low enzyme production, or both.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, as to its organization, and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a graph depicting eleven healthy, asymptomatic, starch tolerant subjects that underwent serial ¹³C isotopically-labeled LDx substrate and ¹³C isotopically-labeled glucose breath testing, over two separate days.

FIGS. 2A-2B. FIG. 2A Normal subject undertook ¹³C-LDx breath testing of oligomer digestion (Day 1) and oxidation that nearly matches glucose oxidation (Day 2). Mean 45′-75′ CGO=93%; FIG. 2B Normal subject undertook, single day, biphasic ¹³C-LDx breath testing of oligomer digestion and oxidation that nearly matches adjust glucose oxidation. Mean 45′-75′ CGO=94%. Demonstrates that the earlier version of the two-day breath test algorithm is reduced to a single day, biphasic test that utilizes a super-dose of ¹³C-glucose as a reference substrate to adjust for habitus and inherent, between subject, metabolic differences.

FIG. 3 is a graph depicting an abnormal subject with irritable bowel syndrome and severe intolerance to ingestion of dietary starches underwent breath testing with ¹³C-LDx Oligomer digestion (Day 1) and oxidation that falls 33% short of reference glucose oxidation (Day 2). Mean 45′-75′ CGO=67% and consistent with an abnormal test result to indicate disaccharidase insufficiency.

FIG. 4 is a graph depicting ¹³C-LDx breath testing of eight, obese female subjects before and eight weeks after gastric by-pass surgery for treatment of refractory obesity. [Change in body mass index: −6.7 (48.6+/−7 vs. 41.9+/−7)]. Mean ¹³C-LDx biphasic breath test results indicate decreased digestive capacity secondary to decreased mucosal enzyme exposure to the dietary starch load.

FIG. 5 is a graph depicting an abnormal subject [AMD, hetrozygous genes for sucrase (3q26.1)p.PHE1745CYS (c.5234 T>G), normal isomatase] 13C-LDx biphasic breath test of oligomer (50 milligrams with 20% unlabled modified starch load), followed by breath sample analyses at 45 minutes elapsed time, 60 minutes elapsed time and 75 minutes elapsed time, and followed by phase II reference ¹³C-glucose breath test using (150 milligrams with 10% unlabeled glucose load). Coefficient of Glucose Oxidation (CGO) for ¹³C-LDx Oxidation: 89.1; [NORMAL TEST: CGO FOR LDx>70].

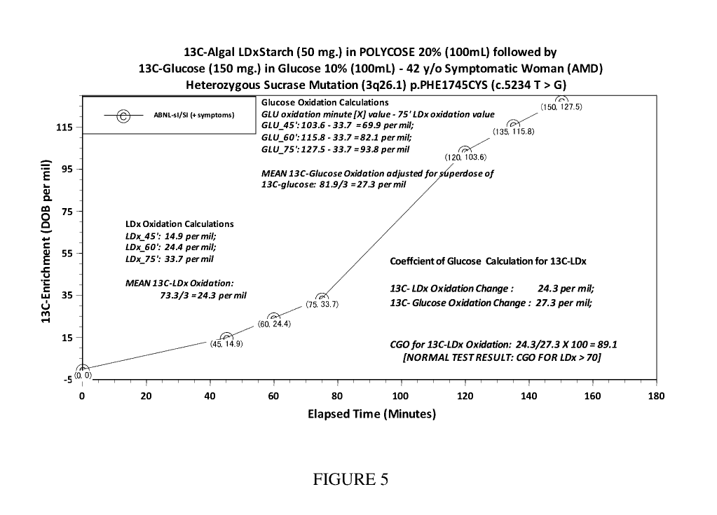

FIG. 6 is a graph depicting an abnormal subject [AED, compound hetrozygous genes for sucrase and isomaltase (3q26.1)L.741P/F1745(c.2278 T>C)] ¹³C-LDx biphasic breath test of oligomer (50 milligrams with 20% unlabled modified starch load), followed by breath sample analyses at 45 minutes elapsed time, 60 minutes elapsed time and 75 minutes elapsed time, and followed by phase II reference 13C-glucose breath test using (150 milligrams with 10% unlabeled glucose load). Coefficient of Glucose Oxidation (CGO) for 13C-LDx Oxidation: 18.7; [ABNORMAL TEST: CGO FOR LDx<70]).

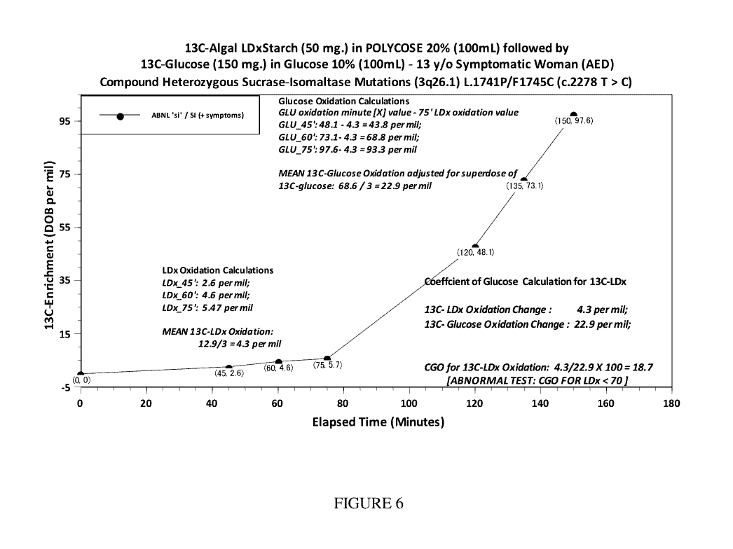

FIG. 7 is a graph illustrating an abnormal subject [EJD, heterozygous gene for isomaltase (3q26.1)L.741P)] ¹³C-LDx biphasic breath test of oligomer (50 milligrams with 20% unlabled modified starch load), followed by breath sample analyses at 45 minutes elapsed time, 60 minutes elapsed time and 75 minutes elapsed time, and followed by phase II reference ¹³C-glucose breath test using (150 milligrams with 10% unlabeled glucose load). Coefficient of Glucose Oxidation (CGO) for ¹³C-LDx Oxidation: 44.9; [ABNORMAL TEST: CGO FOR LDx<70]).

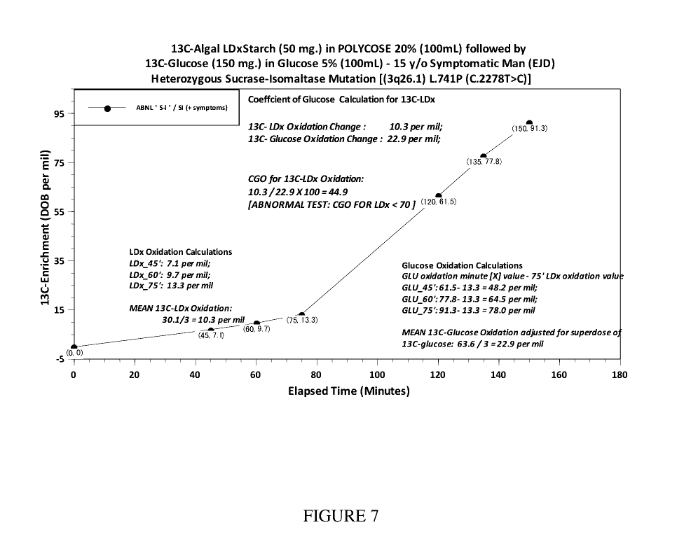

FIG. 8 is a graph depicting phenotyping of mice by starch substrate breath test that demonstrates the effect of MGAM genotype on digestion and oxidation to breath ¹³CO₂ following a ¹³C-α-LDx bolus feeding. The timed mean±SD of ΔOB ¹³CO₂ enrichment by 4 MGAM null (open circles) and 4 WT (filled circles) mice. The points with significant difference by ANOVA and Dunnett's Test at p<0.01 are noted by asterisks.

FIG. 9 is a table illustrating ¹³C-starch hydrolytic response to amyloglucosidase-containing nutritional supplementation (60 mg in 5 capsules, precise activity unknown) in three subjects.

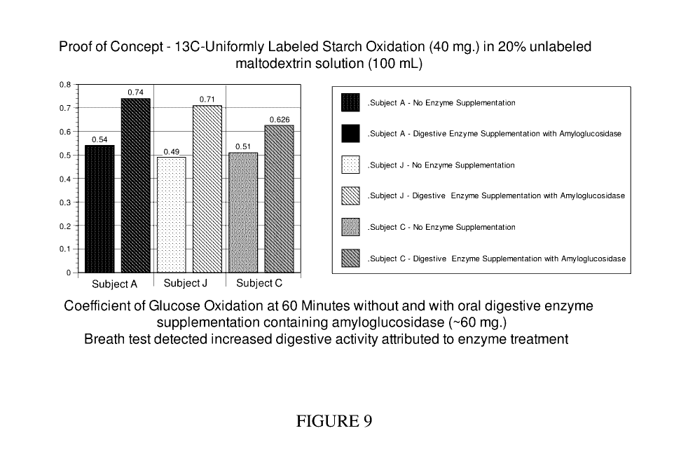

FIG. 10 is a graph that demonstrates the effect of 500 milligrams amyloglucosidase upon the biphasic ¹³C-α-LDx breath test. The patient had relatively normal digestive capacity and the low dose test enzyme only marginally affected the rate of digestion. Results are consistent with the expected physiological response to intragastric digestion and the associated down regulation of gastric emptying (pyloro-duodenal osmotic receptor response to osmotic pressure (Barker et al., 1974, PMID: 4822585). Results indicate that free glucose production in the gastric lumen decreased gastric empyting, thereby reducing appearance of ^{13C}-carbon dioxide in the breath, as represented by the decrease in the LDx-starch coefficient of glucose oxidation (CGO) obtained with concurrent amyloglucosidase enzyme administration [A] (62.2) as compared with control sample CGO (47.8). It is hypothesized that the decrease rate of substrate delivery to the small bowel should equate to overall increased digestion and absorption and decreased symptoms that could be attributed to malabsorption and distal fermentation in the colon. The breath test outcome is silent with regard to efficacy for symptom relief.

FIGS. 11A-11D. Results of four genetically SI-characterized subjects who undertook the ¹³C-S/GBT (gastroscopic breath testing) with simultaneous breath and blood sampling to validate outcomes. FIG. 11A 45.9 year-old healthy, asymptomatic woman without suspected SI gene mutation and normal CGO for ¹³C-sucrose (>80%); Chromosome 3-NC_000003.12; Chr. 3 q25 2-26.2; WT/WT. FIG. 11B 23.3 year-old symptomatic woman with heterozygous SI gene mutation and abnormal mean CGO for ¹³C-sucrose (<80%); Chromosome 3-NC_000003.12; Chr. 3 q25 2-26.2; [c.3218G>A(p.G1073D)/WT]. FIG. 11C 53.9 year-old symptomatic woman with heterozygous SI gene mutation and abnormal mean CGO for ¹³C-sucrose (<80%); Chromosome 3-NC_000003.12; Chr. 3 q25 2-26.2; [c.5243T>G(p.P1745C)/WT]. FIG. 11D 46.7 year-old symptomatic woman with homozygous SI gene mutation and abnormal mean CGO for ¹³C-sucrose (<80%); Chromosome 3-NC_000003.12; Chr. 3 q25 2-26.2; [c.3218G>A(p.P1073D)/c.3218G>A(p.G1073D)].

FIG. 12 is a graph depicting the relationship between plasma ¹³C-enrichment and breath ¹³C-enrichment in experiments on the four subjects with various sucrose-isomaltase genotypes depicted in FIGS. 11A-11D.

FIG. 13 is a graph depicting the regression analysis of data obtained from two subjects (X & Y) with distinctive sucrase-isomaltase genotypes and symptoms. Subject X had a mutation in the isomaltase domain, was starch/oligosaccharide intolerant (typical gastrointestinal symptoms) and had a abnormally low mean 60′-75′ coefficient of glucose oxidation value of 0.66; consistent with the heterozygous condition and maldigestion. Subject Y had a mutation in the sucrase domain, was starch/oligosaccharide tolerant (minimal gastrointestinal symptoms) and had a normal mean 60′-75′ coefficient of glucose oxidation value of 0.99; only consistent with the heterozygous condition and maldigestion of sucrose, not starch. Correlation of Determination: 0.75.

### DETAILED DESCRIPTION OF THE INVENTION

### Definitions

As used herein the specification, “a” or “an” may mean one or more. As used herein the claim(s), when used in conjunction with the word “comprising”, the words “a” or “an” may mean one or more than one. As used herein “another” may mean at least a second or more. Furthermore, as used herein, the terms “including”, “containing”, and “having” are open-ended in interpretation and interchangeable with the term “comprising.”

As used herein the term “α-glucosidase” as used herein represents a descriptor inclusive of all mucosal oligomer digestion activities including maltase, isomaltase (measured as palatinase) and glucoamylase activities. A “uniformly labeled substrate” is a biological molecule that contains an elemental atom isotope species of interest, where each atom species will appear and be dispersed in the substrate molecule equally (uniformly), such that the species atom isotope could be used as a marker or tracer for which the substrate will undergo metabolism and some or all of the tracer atom isotope will be incorporated into the molecular structure of the resultant compound in consistent portions. For example, glucose is a typical biological substrate, and each molecule of uniformly-labeled glucose includes 6 carbon atom species that may contain carbon isotope ¹²C or carbon isotope ¹³C and carbon species and all loci are equally proportioned to either carbon isotope ¹²C or carbon isotope ¹³C. As a further example, the number 1 carbon atom in a selected molecule may have a 99% chance of being a carbon isotope ¹³C and match carbon atoms numbers 2, 3, 4, 5, and 6 for 99% enrichment, each. Therefore, all 6 carbon atoms in that selected molecule are uniformly labeled and the combined atomic mass weight of that selected molecule would be 186 Daltons; this accounts for each additional atomic mass unit associated with each carbon atom. Conversely, if another selected glucose molecule were to be assembled from two smaller molecules, each with differing proportions of carbon atom isotope ¹²C and carbon atom isotope ¹³C, the resultant glucose molecule would have highly variable portions of carbon atom isotope ¹³C scattered at the various carbon loci and would not be considered uniformly labeled.

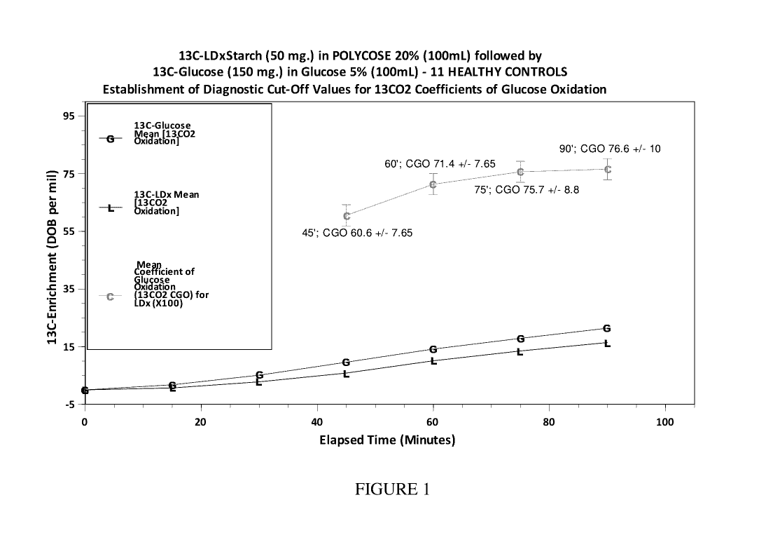

As used herein the terms “¹³C-α-limit dextrin” or “¹³C-α-LDx” refers to at least one uniformly labeled ¹³C-α-limit dextrin or a mixture of uniformly labeled ¹³C-α-limit dextrins that are soluble polymers comprised of glucose units produced from a ¹³C-containing starch that has been treated by mammalian α-amylase or another amylase enzyme to essentially complete endoglucosidase enzyme hydrolysis. When referring to the terms “^{x}C-α-limit dextrin” and/or “^{x}C-α-LDx”, the term “x” represents the number 12, 13 or 14, and the terms “¹²C-α-LDx, ¹³C-α-LDx” and “¹⁴C-α-LDx” represent a substrate having carbon atoms with atomic masses of 12, 13 and 14 atomic mass units respectively. ¹³C is a naturally occurring stable isotope of ¹²C. ¹⁴C is a radioactive isotope. Specifically, the terms “¹³C-α-limit dextrin” or “¹³C-α-LDx” denote a glucose polymer with a measurable proportion of the carbon atoms being ¹³C that can only be assimilated after digestion to glucose by small intestinal, mucosal enzymes, unless malabsorbed and pass to the colon, with time, where fermentation by flora can occur. ¹³C-Maltodextrins are a group of soluble polymer having 100% glucose units produced from any ¹³C-containing starch that has been treated by mammalian α-amylase or other amylase enzyme such that the resulting treated polymer has a dextrose equivalent between 3 and 20. Dextrose equivalent (DE) is a measure of the amount of reducing sugars present in a sugar product, relative to dextrose (a.k.a. glucose), expressed as a percentage on a dry basis. Native starch molecules have only one reducing end. Maltodextrins have multiple reducing ends and are compared to free glucose (dextrose) with 100% reducing ends. Commercial maltodextrins have a range of DE of about 20% whereas commercial corn syrups have greater than 80% DE (See FIG. 1 of Quezada-Calvillo R et al., “Luminal Substrate “Brake” on Mucosal Maltaseglucoamylase Activity Regulates Total Rate of Starch Digestion to Glucose” J Pediatr Gastroenterol Nutr. 2007 in which the contents of this reference are incorporated herein by reference in its entirety).

The term “Coefficient of Glucose Oxidation” or “CGO” provides normalization of the starch digestion, absorption and oxidation by its product glucose-adsorption and oxidation. This CGO is very important in infants and children where glucose oxidation decreases with age, concomitant conditions of stress and may be important in disorders of glucose homeostasis at any age (See Robayo-Torres et al., “¹³C-breath tests for sucrose digestion in congenital sucrase isomaltase-deficient and sacrosidase supplemented patients.” J Pediatr Gastroenterol Nutr. 2009; 48:412-418 in which the contents of this reference are incorporated herein by reference in its entirety).

“Effective amount,” “therapeutically effective amount” or “pharmaceutically effective amount” means that amount which, when administered to a subject or patient for treating a disease, is sufficient to effect such treatment for the disease.

The above definitions supersede any conflicting definition in any of the references that are incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### GENERAL EMBODIMENTS

Carbohydrates (sugars and starches) contribute approximately 70% of the calories in the normal diet. The final step in absorption of carbohydrates is for brush border enzymes to convert carbohydrates into single glucose, galactose or fructose units that the body can absorb. There are enzymes (disaccharidases) that convert disaccharides into single units, and the lack of disaccharidases results in lactose (milk sugar) and sucrose (table sugar) intolerance and are well-known causes of chronic diarrhea in young children and, at older ages, dyspepsia. Glucoamylase is a lesser-known enzyme responsible for the last stages of starch digestion. Some dyspeptic children have intestinal glucoamylase deficiency. Primary intestinal enzyme deficiency has been linked to congenital and adult type lactose intolerance, congenital sucrase-isomaltase deficiency, primary starch intolerance (maltase deficiency), secondary disaccharidase deficiency due to mucosal diseases such as viral diarrhea, malnutrition, celiac disease, or HIV infection. Also, intestinal enzyme deficiency has been linked to: pre-transcriptional congenital deficiencies, post-transcriptional congenital deficiencies, viral-induced mucosal injury (poliovirus, rotavirus and norovirus), bacterial-induced injury (salmonella), parasitic infestation (giardiasis and cryptosporidiosis), celiac disease, food allergies, idiopathic eosinophilic inflammatory disease, regional enteritits (inflammatory bowel disease), infiltrative disease (amyloidosis), toxic radiation exposure, drug-induced injury (anti-neoplastic chemotherapy), nonsteroidal anti-inflammatory medication injury (COX inhibitors) and antimicrobial agents (macrolides). Specifically, there is a need for a non-invasive isotopically-labeled ¹³C-starch or ¹³C-α-limit dextrin (¹³C-α-LDx) breath test to detect intestinal glucoamylase deficiency and/or abnormal brush border intestinal enzyme activity.

Brush border intestinal α-glucosidases are responsible for the final stages of proximal starch digestion before absorption can occur. It was shown that 21% of children evaluated for FGID have low α-glucosidase activity, which could be a cause for their symptoms (Karnsakul et al., 2002). This is analogous to the relationship between lactase (β-glucosidase) insufficiency and lactose intolerance, but starches are diverse molecules, requiring a multi-step digestive process and, as such, are more difficult to study. As stated above, starches provide the majority of calories in the typical Western child's diet and are nearly impossible to restrict from the diet when problematic. In this example, a subset of children with FGID and who also have intestinal α-glucosidase insufficiency would be provided oral fungal-derived α-glucosidase replacement therapy (AMG). It is expected that most affected children will respond to test therapy as measured by improvement in symptoms and increased substrate oxidation breath test (BT) scores, represented by increased coefficient of glucose oxidation.

Whole ¹³C-starch breath test may be used to measure glucosidase deficiency and assess the outcome of therapy aimed at improving starch digestion. Under normal conditions, the glucose derived from the enzyme-digested ¹³C-starch is metabolized (oxidized) and appears in the breath as labeled ¹³C-carbon dioxide which can be measured using a desktop instrument. Conversely, if the starch cannot be digested in the upper small intestine, undigested starch or partially digested glucose oligomers derived from the starch are confined to the intestinal lumen and pass into the colon where intestinal bacteria can metabolize the starch. This process often produces symptoms associated with enzyme deficiency ailments. In the event of poor absorption, the isotope label is reduced in breath samples taken during digestion when compared to simple reference substrate oxidation. This principle is also applicable for other less-complex isotopically-labeled substrates that require mucosal hydrolysis including ¹³C-limit dextrins, ¹³C-oligosaccharides, 13C-peptides, and ¹³C-fatty acids. It was found that a comparative reference test using ¹³C-glucose is needed to compensate for individual variations. The results of the two tests (expressed as a calculated coefficient of the two sub-test outcomes, the coefficient for glucose metabolism for the target substrate in question) is indicative of some problems in digestion occurring at the intestinal mucosal level.

The coefficient for LDx digestion and subsequent glucose metabolism is defined as the ratio of ¹³C-carbon dioxide breath enrichment of uniformly-labeled ¹³C-LDx digestion to the ¹³C-carbon dioxide enrichment derived from uniformly-labeled ¹³C-glucose digestion. In some embodiments, the uniformly-labeled ¹³C-glucose is used as a reference substrate [coefficient of glucose oxidation (CGO)]. Glucose absorption is independent of intestinal brush border maltase-glucoamylase (MGAM) activity whereas starch digestion (test substrate) is dependent on isomaltase and/or MGAM enzyme activity prior to uptake. When starch digestion is impaired, the ratio of the test and reference values decreases (about 80% or greater CGO appears normal). When glucose absorption is abnormal, both starch and glucose oxidations will be reduced (Lifschitz et aI., 1989).

Breath test substrates are prepared and labeled appropriately for clinical investigations. For example, the isotopically labeled substrate is diluted in non-isotopically labeled soluble starch or malto-dextrins (20 grams), suspended as a flavored aqueous beverage and pasteurized. The substrates are food-stuffs in the GRAS (Generally Regarded As Safe) category, and the substrates, ¹³C-glucose and ¹³C-starch, are non-radioactive. As such the substrates are not subject to Nuclear Regulatory Commission regulations. The whole crude starch is commercially available and has a uniform ¹³C-atom enrichment of at least 95%. The uniform ¹³C-glucose is commercially made from the same labeled starch or synthesized. Breath samples are collected using foil-lined breath collection bags that contain a one way valve mouthpiece, though less robust, plastic breath collection bags are also available. The breath sample bags, when double sealed, transport easily for analysis or can be analyzed immediately at the bedside using an analyzer instrument.

Dyspepsia is defined as a persistent or recurrent pain or discomfort in the upper abdomen. The prevalence of dyspepsia in children is reported as approximately 10% (Hyams, 1996). This entity also known as chronic recurrent abdominal pain or functional abdominal pain in children. It a common malady in both children and adults and it is often suspected to be of functional origin due to stress or anxiety. In a preliminary study, 226 children underwent endoscopies because of dyspeptic symptoms. Forty-four children of this group were studied with biopsies to determine a possible role of deficient disaccharidase activities in dyspepsia (Karnsakul et al., 2002). Duodenal glucoamylase, sucrase and lactase activities were assayed. Twenty-two (50%) of the study children had low activity of one or more disaccharidases. Twelve subjects (28%) had low activity of glucoamylase. Combined low activity of glucoamylase, sucrase and lactase was observed in eight subjects (Karnsakul et al., 2002; and, Nichols et al., 2002).

Complaints of recurrent abdominal pain represent a common reason for pediatrician office visits, and account for about 20% of referrals to pediatric gastroenterologists and may be a contributing factor to irritable bowel disease in adults (Pasricha P J. Approach to the patient with abdominal pain. In: Yamada T, Alpers D H, Kaplowitz N, et al., eds. Textbook of Gastroenterology. 4th ed. Philadelphia, Pa.: Lippincott Williams and Wilkins; 2003:781-801). The ¹³C-Labeled Starch/Alpha Limited Dextrins Digestion Breath Test for Intestinal Brush Border Intestinal Glucoamylase Activity as disclosed herein may be used to identify symptomatic patients who are enzyme deficient and indicate those who could benefit from confirmatory gastrointestinal endoscopy with biopsies. Afflicted patients might benefit from dietary restrictions or supplementation of digestive enzymes (amyloglucosidase, lactase, and sucrase, which are approved for use as dietary supplements by the FDA [GRAS Notices 000088-90, Apr. 3-4, 2002]).

Starch-derived alpha LDx digestion results in production of glucose that may be directly absorbed and metabolized, and ultimately results in carbon dioxide that is detectable in the breath. The single day, biphasic breath test disclosed herein comprises the following general steps. First, a baseline breath sample is obtained (approximately 2 liters) in a gas-tight collection bag and held as a reference gas for subsequent comparisons to determine changes in isotopic enrichments. Subsequently, a starch or LDx oligosaccharide substrate labeled with the stable isotope (¹³C) or radioactive isotope (¹⁴C) carbon atoms is administered to a subject as a dose of at least 50 milligrams in an excipient aqueous solution, of 100 milliters, containing 20 grams of unlabeled glucose dextrins and polymers. The labeled whole starch or LDx oligosaccharide substrate is metabolized which results in production of isotopically labeled carbon dioxide that appears and is detectable in the breath of a subject. The isotopically-labeled carbon dioxide that is derived from the labeled starch is sampled at three elapsed time points (45 minutes elapsed time, 60 minutes elapsed time, 75 minutes elapsed time, for example) and measured as a comparative difference to the baseline breath sample. Each resultant value is recorded as a change in enrichment and the mean change in enrichment is calculated. Likewise, during phase two, a labeled glucose substrate, at a super-dose of at least three-times or five-times the dose of labeled LDx oligosaccharide substrate, which is composed of stable isotope (¹³C) or radioactive isotope (14C) carbon atoms, in an excipient aqueous solution of 100 milliliters containing 10 grams of unlabeled glucose, is administered to a subject. The labeled glucose is directly absorbed without mucosal enzyme action and internally metabolized which results in isotopically labeled carbon dioxide that is detectable in the breath. The isotopically-labeled carbon dioxide that is derived from the labeled glucose is sampled at three elapsed time points (45 minutes elapsed time, 60 minutes elapsed time, 75 minutes elapsed time) and measured as a comparative difference to the original baseline breath sample. Each resultant value is recorded as a change in enrichment and the mean change in enrichment is calculated. The ¹³CO₂ measurement proportionally attributable to the labeled glucose is established after subtracting the mean LDx oligosaccharide enrichment value measured at the respective 75-minute time point and mathematically dividing the resultant value by the super-dose factor, either 3 or 5, and compared to the ¹³CO₂ measurement resulting from the isotopically labeled LDx oligosaccharide or whole starch and the ratio of the two respective substrate change in isotopic enrichments is calculated and recorded as the “coefficient of glucose oxidation for LDx oliosaccahride” or “coefficient of glucose oxidation for whole starch. In alternate embodiments, sucrose or lactose (disaccharides), in lieu of LDx oligosaccharide or starch, that is composed of stable isotope (¹³C)(¹³C) or radioactive isotope (¹⁴C) carbon atoms is metabolized which result in isotopically labeled carbon dioxide that is detectable in the breath of a subject and the coefficient of glucose oxidation for the alternative substrate, sucrose or lactose (disaccharides). In additional and alternate embodiments, breath samples, collected before ingestion of the labeled substrate (starch or disaccharides) and periodically after ingestion of substrate can be analyzed using infrared mass-dispersive spectrometry or gas isotope ratio mass spectrometry (¹³C) or scintillation counting (¹⁴C) to determine the enrichment of isotope derived from test substrates. The ratio of carbon dioxide isotope enrichment derived from starch or disaccharides divided by the isotope enrichment derived from glucose is diagnostic of intestinal glucoamylase, maltose-glucoamylase or disaccharidase deficiency or absence and that intestinal maltose-glucoamylase or disaccharidase deficiency enzyme deficiency is a cause of symptoms. A difference in isotopic enrichment outcomes between starch and LDx oligosaccharides, whereas the oxidation enrichment attributable to LDx is greater than the oxidation enrichment attributable to whole starch, describes the component attributable to pancreatic alpha-amylase activity and mucosal MGAM activity.

To understand the role of α-glucosidase activities in the assimilation of complex dietary carbohydrates and demonstrate their importance in health and disease, it is necessary to identify patients with FGID and “insufficient” duodenal mucosal α-glucosidase activities with assay of endoscopically-obtained biopsies and test digestion of partially hydrolyzed, stable isotopically (¹³C) labeled starch-derived oligomers [¹³C-α-limit dextrins and maltodextrins] and sucrose, develop treatment strategies, and therefore address the nutritional and metabolic consequences of chronic duodenal α-glucosidase insufficiency.

Though no longer universally endorsed, assessment of FGIDs remains the most commonly stated indication for esophagogastro-duodenoscopy (EGD) in child (Gilger and Gold, 2005) and adult populations (Lieberman et al., 2004). The PEDS-CORI (Pediatric Endoscopy Data System Clinical Outcome Research Initiative) reported, during the first 4 years, a total of 17,457 EGD procedures. Almost half (44%) of all childhood EGD investigations found no obvious pathology on inspection and biopsy histology (Gilger and Gold; 2005). Among the 18,444 EGD reports provided by gastroenterologists to the adult CORI data bank (Taylor et al., 2000) the most common report was no abnormality detected by inspection or biopsy pathology. Because most EGD results are uninformative, they serve as a source of frustration when a definitive diagnosis was not made. Childhood AP-FGIDs have been predictive or linked to adult IBS (Walker et al., 2004; and Russo et al., 2004). Earlier guidelines had recommended that all children with suspected symptomatic FGID have EGD as part of the clinical evaluation (Hyams, 2004). There are clinical groups that diagnose in excess of 500 FGID cases per year.

Identification of suitable children/subjects with FGID requires that they are mature enough to provide an accurate history of their pain and other symptoms. A validated questionnaire for parents of children 4 to 9 years of age and another (self-reported) for children 10 years of age and older [the Rome II Questionnaire on Pediatric GI Symptoms (QPGS)] have been used to aid in diagnosis and classification (Caplan et al., 2005). Because of cultural and regional influences, it would be difficult to utilize a single symptom index developed and validated in one region to be indiscriminately applied to another region and to expect the outcome data to be valid. An additional instrument, called the Malaty Multi-dimensional Measure for Recurrent Abdominal Pain or MMM-RAP (Malaty et al., 2005) has recently been developed and validated for children. It evaluates 4 dimensions: (1) pain intensity (evaluative variables: Wong-Baker face; worst pain in 3 months; and average pain in 3 months); (2) non-pain symptoms (evaluative variables: nausea/vomiting; loss of appetite; heartburn; diarrhea; constipation/hard stool; bloating/abdominal distention; passing gas; burning/belching; problem with ingestion of milk; sleep problem; bad breath; and sour taste), (3) pain disability (evaluative variables: missed school days; daily activities; and week-end activities), and (4) satisfaction with state of health, and has a good level of internal consistency for each scale (>0.70). This instrument is most amendable to assessment of shorter-term responses to therapy. Together these two instruments provide a good assessment of childhood IBS and FAP.

There is strong evidence to indicate that a significant percentage of FGID cases are related to starch maldigestion secondary to mucosal disaccharidase insufficiency, including isomaltase and glucoamylase. This warrants development of effective treatment for these FGID cases and the need to assess such treatments. Critical to the development of an effect treatment for these FGID cases is a better understanding and interpretation of isotopically-labeled breath test outcomes. To show that there is a cause-and-effect relationship between α-glucosidase insufficiency and symptoms, one may assess symptom scores, biopsy assays ¹³C-starch and ¹³C-α-LDx breath test outcomes and by evaluating the clinical response to orally administered AMG, a potent α-glucosidase with both α-1-4 and α-1-6 hydrolytic activities. This may have a significant and favorable impact upon this large target population, and perhaps may have a beneficial effect other conditions, such as Crohn's disease, post-viral syndromes and chronic HIV/AIDS infection (Taylor et al., 2000).

The interest and work in the area of intestinal brush border carbohydrate hydrolytic enzyme activity began during the 1980s. At that time the search was for factors that would speed mucosal enzyme function recovery following rotaviral-induced malabsorption (Calvin et al., 1985). Results indicated that 22-35% of all abnormal biopsy samples had abnormal range-specific disaccharidase activities. It was also found that breast milk had no beneficial effect (Shulman et al., 1989). Lebenthal et al., confirmed the observation that intestinal glucoamylase insufficiency is present in some patients with chronic diarrhea (Lebenthal et al., 1994). It was then realized that a thorough understanding of brush border physiology was lacking and it was desirable to pursue experiments to characterize two main brush border enzymes other than lactase, SI and MGAM.

It was reported that mosaic expression of brush-border enzyme proteins existed as a result of the pathological lesions of the mucosa in infants with chronic diarrhea (Nichols et al., 1998). Subsequently, it was hypothesized that, after luminal amylytic activity, MGAM plays a unique role in the digestion of malted dietary oligosaccharides. Human small intestinal MGAM cDNA was cloned to permit study of the individual catalytic and binding sites for maltose and starch enzyme hydrolase activities in subsequent expression experiments (Nichols et al., 1998). Human MGAM was purified by immunoisolation and partially sequenced. MGAM cDNA was amplified from human intestinal RNA using degenerate and gene-specific primers with the reverse transcription-polymerase chain reaction. The 6,513-base pair cDNA contains an open reading frame that encodes an 1,857-amino acid protein (molecular mass 209,702 Da). MGAM has two catalytic sites identical to those of SI, but the proteins are only 59% homologous. Both are members of glycosyl hydrolase family 31, which has a variety of substrate specificities. The findings indicated that divergences in the carbohydrate binding sequences must determine the substrate specificities for the four different enzyme activities that share a conserved catalytic site.

Shortly thereafter, the case of an infant with biopsy-assay-proven pan-disaccharidase insufficiency was investigated (Nichols et al., 2002). Sequencing of MGAM cDNA revealed homozygosity for a nucleotide change (C1673T) that caused an amino acid substitution (S542L) 12 amino acids after the N-terminal catalytic aspartic acid. Clinical testing with a prototypical ¹³C-starch breath, ¹³CO₂-loading test affirmed proximal starch malabsorption. The introduction of this mutation into “wildtype” N-terminus MGAM cDNA was not associated with obvious loss of MGAM enzyme activities when expressed in COS 1 cells. This amino acid change was subsequently found in other people. Sequencing of the promoter region revealed no nucleotide changes. In contrast, organ culture experiments showed that MGAM, lactase and SI were each normally synthesized and processed, indicating a complex etiology underlying the clinical insufficiency. This raised additional questions, including the frequency of disaccharidase activity among infants and children being evaluated for enigmatic functional abdominal pain. The lack of evidence for a causal nucleotide change in the MGAM gene in these patients and the concomitant low levels of lactase and sucrase activity suggested that the depletion of mucosal MGAM activity and starch digestion is caused by shared, pleiotropic regulatory factors.

Disaccharidase insufficiency is prevalent in children that present for evaluation of FGID. Through a survey study of 44 children that presented for EGD for evaluation of FAP, it was found that 22 children had low specific activity of one or more glucosidases (Karnsakul et al., 2002). Twenty-seven percent (12 of 44) had low glucoamylase activity that coincidently matched the post-rotaviral findings from two decades earlier. Eight subjects had low activities of glucoamylase, sucrase and lactase. Low glucoamylase activity was not correlated with the isoform phenotype of MGAM as described by metabolic labeling and sodium dodecyl sulfate electrophoresis. Novel nucleotide changes were not detected in one subject with low glucoamylase activity or in two subjects with variant isoforms of MGAM peptides. This indicated that the problem with disaccharide insufficiency is more widespread than previously recognized, and it was not clear how long impaired disaccharidase activity persists after initial injury.

In vivo and in vitro studies were carried out to understand the role of MGAM in mammalian starch digestion (Quezada-Calvillo et al., 2007). Genetic knock-out mice were produced that failed to express products of the MGAM gene, and a small animal isotopic breath collection device was constructed to collect isotopically-labeled breath test (BT) samples. This study affirmed the important role that MGAM plays in starch digestion and, more specifically, in low-starch diets, given the low Km for the enzyme. It was also revealed that SI has a high Km and is responsible for slow glucogenesis and that alpha-amylase works synergistically with MGAM and SI, but that alpha-amylase cannot completely hydrolyze starch to glucose. This served to underscore the utility of isotopically-labeled breath testing to assess enzymes other than lactase.

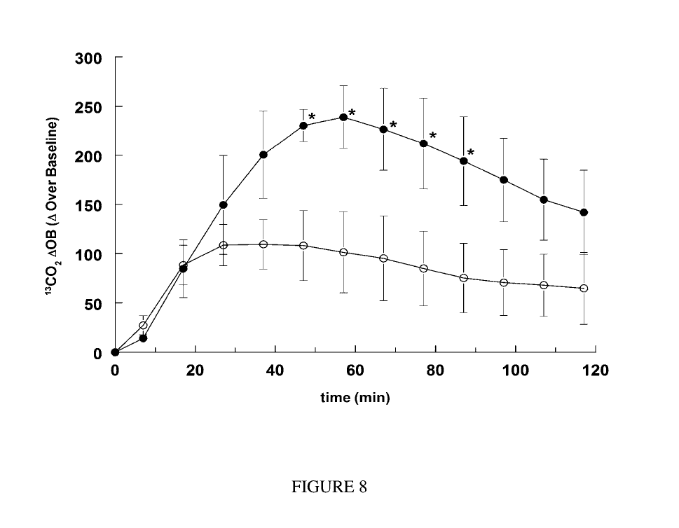

FIG. 8 shows the phenotyping of mice by whole ¹³C starch substrate breath test. FIG. 8 also shows the effect of MGAM genotype on digestion and oxidation to breath ¹³CO₂ following a ¹³C-α-LDx bolus feeding. FIG. 8 also shows the timed mean±SD of DOB ¹³CO₂ enrichment by 4 MGAM null (open circles) and 4 WT (filled circles) mice. The points with significant difference by ANOVA and Dunnett's Test at p<0.01 are noted by asterisks.

In a previous study, the total starch digestive capacity of the small intestinal mucosa and specified the role of SI and MGAM has been determined (Quezada-Calvillo et al., 2007). Using endoscopically-obtained biopsy samples, immunoprecipitation was used to isolate MGAM and SI and maltodextrin was used as the substrate to assess products of digestion. MGAM has a much lower Km (higher glucogenic capacity) than SI despite the fact that MGAM is less active in hydrolyzing maltodextrin. MGAM is a faster glucose producer, but is inhibited by excess maltodextrin. The term “Luminal Starch Substrate Brake,” relates to an evolutionary phenomenon and it is proposed that during times of low free starch in diet (such as early man), MGAM historically played a significant role in starch digestion. But now, in modern times, since the dietary non-resistant starch and free oligosaccharide content, including maltose, sucrose and maltodextrins, is much higher, liberated dextrins may inhibit MGAM to allow SI, with its higher Km, to slowly release food glucose for assimilation and increase the propensity for maldigestion. Although the assertion, outlined above, matches the data in the study, it is not substantiated by enough external data, and thus will require further study. It is unclear if the tremendous amounts of free sucrose in the Western diet exceed luminal hydrolytic capacity and lead to “functional” symptoms of abdominal distress in “normal” people. In a word, concentrated, refined table sugar, simple oligosaccharides and dextrins are not the optimal source of carbohydrate-derived calories, and excess dietary content pushes the brush-border disaccharidase system to the max. Furthermore, the relationship between malabsorbed oligosaccharides, luminal brake activity and motility is unknown, and further work is planned. Likewise, the dynamic interaction between dietary content, digestive capacity, satiety and the obesity epidemic is not well understood.

The function of brush border disaccharidases was further elucidated to challenge the notion that brush border MGAM and SI only act on starch after it is broken down by salivary and pancreatic amylase (Ao et al., 2007). Using recombinant MGAM to examine activity on starch hydrolysis, in vitro, it was reported that the rate and extent of hydrolysis of amylomaize-5 and amylomaize-7 by MGAM to glucose were greater than for other starches. Such was not observed with fungal amyloglucsosidase (AMG) or pancreatic alpha-amylase. The degradation of native starch granules showed a surface-furrow pattern in random, radial or tree-like arrangements that differed substantially from the erosion patterns of AMG or alpha-amylase. As such, pancreatic alpha amylase may not be absolutely necessary for starch digestion within the human small intestine in that it plays a role that augments brush border enzyme function. Multiple factors, such as starch molecular structure, osmotic effect of unprocessed starch, presentation of starch to the brush border enzyme, and starch effect on gastric motility, must be examined before these assumptions can be incorporated into the full schema.

A diagnostic BT for active Helicobacter pylori infection that detects changes in expired ¹³CO₂ after oral administration of ¹³C-urea has been developed (Graham et al., 1987). Extending this work to further understand the syndrome of CSID, a clinical study was performed to optimize the diagnosis and treatment approach (Robayo-Torres et al., 2009). Specific diagnosis requires upper gastrointestinal biopsy with evidence of low-to-absent sucrase enzyme activity and normal histology. The hydrogen BT is useful, but is not specific for confirmation of CSID.

A more specific ¹³C-sucrose labeled BT was investigated and it was found that CSID could be detected noninvasively, without duodenal biopsy sucrase assay, and the ¹³C-sucrose BT could document restoration of sucrose assimilation with oral supplementation with yeast-derived sacrosidase. Twenty pediatric patients were studied: 10 with CSID diagnosed by low biopsy sucrase activity and 10 who underwent endoscopy and biopsy for functional abdominal pain or chronic diarrhea that had normal mucosal enzyme activity and histology. Uniformly labeled ¹³C-glucose and ¹³C-sucrose loads were orally administered. ¹³CO₂ breath enrichments were assayed using an infrared spectrophotometer. In CSID patients, the ¹³C-sucrose load BT was repeated adding oral sacrosidase. Sucrose digestion and oxidation were calculated as a mean percent coefficient of glucose oxidation (CGO) averaged between 30 and 90 minutes. CGO is calculated since the rate of glucose absorption is theoretically not affected by mucosal enzymatic hydrolysis. Its rate of enrichment represents the maximum physiologic assimilative capacity for each subject at each time point and therefore adjusts for individual variations in habits. Paired BTs are always performed back-to-back to minimize confounding variables, but at least 12 hours apart to avoid isotope carry-over (wash-out). In some examples, tests are usually done in the morning.

Classification of patients by ¹³C-sucrose BT percent coefficient of glucose oxidation agreed with duodenal biopsy sucrase activity. The BT also documented the return to normal of sucrose digestion and oxidation after supplementation of CSID patients with yeast-derived sacrosidase.

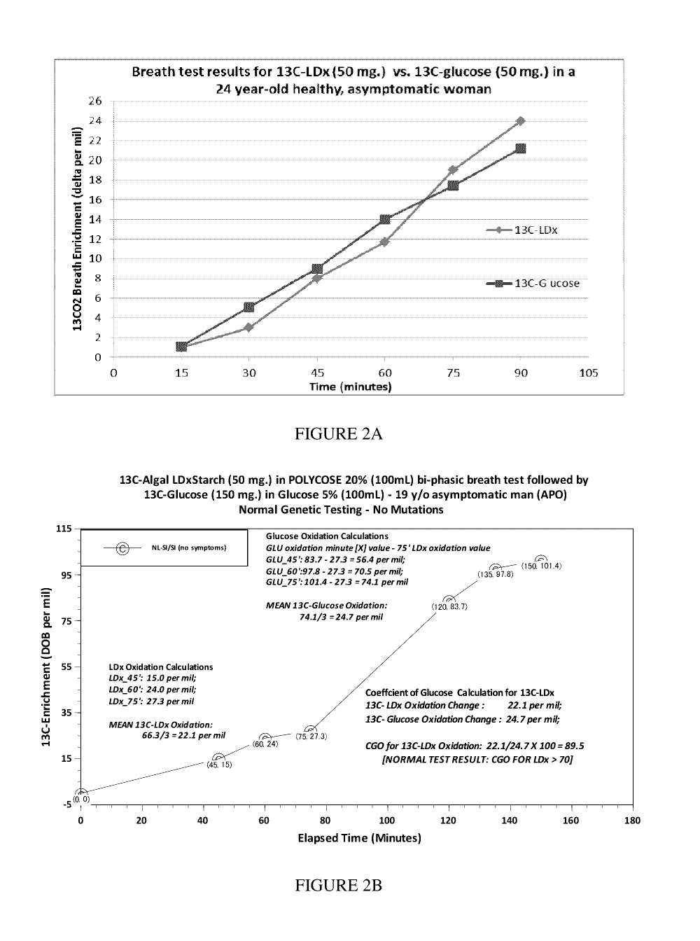

In a way similar to how pancreatic insufficiency is treated, a pilot experiment was carried out with three of the investigators as subjects to determine the potency of commercial AMG supplements on human ¹³C-Starch (edible algal) BT. Commercial OTC supplements make no claims as to any therapeutic efficacy for diagnosed clinical conditions. It was supplemented with food grade AMG from A. niger because this enzyme has been extensively characterized, is widely used in food processing industries, and is formulated and sold in enzyme supplement formulations over-the-counter (OTC). To adjust for individual habits, each subject performed a baseline ¹³C-glucose BT with identical attributes to the ¹³C starch BT. Starch hydrolysis results are reported as a ratio of baseline glucose oxidation (CGO) to test/target substrate oxidation. In this experiment the participating investigators conducted ¹³C-ST BT on separate days; 40 mg of ¹³C-starch was given with and without supplementation by taking six over-the-counter capsules with 12 mg of AMG each by mouth. The ¹³C-starch CGO responses to 72 mg of AMG, supplemented in 6 Source Natural's capsules, (M±SD) revealed baselines, with no AMG supplement of 51±3% vs. AMG supplemented of 76±6%; (a 49% increase, p=0.003, ANOVA). It has been confirmed that these ¹³C-starch BT responses with other brands, but not all OTC AMG supplement brands were active in our mouse AMG experiments; therefore, some were rejected. Since this experiment was done, a more specific isotopically-labeled substrate has been developed, ¹³C-α-limit dextrins (¹³C-α-LDx). FIG. 2 shows the ¹³C-starch hydrolytic response to enzyme supplementation in three subjects. ¹³C-α-limit dextrins

Compositions of the present disclosure may be made using the methods described below. These methods can be further modified and optimized using the principles and techniques of organic chemistry and/or biochemistry as applied by a person skilled in the art. Such principles and techniques are taught, for example, in March's Advanced Organic Chemistry: Reactions, Mechanisms, and Structure (2007), which is incorporated herein by reference.

In general, ¹³C-α-limit dextrins are produced by digesting branched amylopectin starch to short chain oligosaccharides that are vulnerable to incomplete alpha 1-4 bond hydrolysis. For example, universally labeled ¹³C-starch of algal source was exhaustively digested with porcine AMY (Type VI-B) to yield the ¹³C-α-limit dextrins. The resulting ¹³C-α-limit dextrins are insensitive to further luminal digestion.

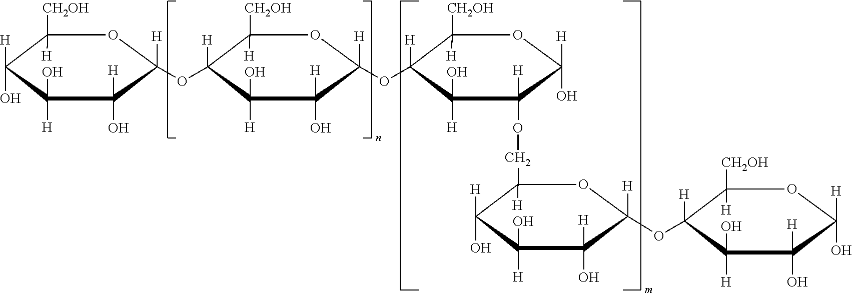

The general formula as shown above represents a uniformly labeled ¹³C-α-limit dextrin or ¹⁴C-α-limit dextrin. In some examples, the ¹³C-α-limit dextrin is a short polymer with approximately 20 glucose units and has very few 1-6 bond linkages. In other examples, the ¹³C-limit dextrin is a short polymer with approximately 12 glucose units and has 3 or 4 1-6 bond linkages. In some examples, the ¹³C-limit dextrin is a mixture of ¹³C-limit dextrins that have 12 glucose units and wherein the mixture has between 20 and 40% 1-6 bond linkages, between 25 and 35% 1-6 bond linkages, or between 28 and 33% 1-6 bond linkages. In some examples, the ¹³C-α-limit dextrin has between 5 and 60 glucose units, 5 and 45 glucose units, 5 and 40 glucose units, 5 and 35 glucose units, 5 and 30 glucose units, 5 and 25 glucose units, 5 and 20 glucose units, 5 and 15 glucose units, 5 and 10 glucose units, 5 and 8 glucose units, 5 and 7 glucose units, 10 and 60 glucose units, 15 and 60 glucose units, 20 and 60 glucose units, 25 and 60 glucose units, 30 and 60 glucose units, 35 and 60 glucose units, 40 and 60 glucose units, 45 and 60 glucose units, 50 and 60 glucose units, 55 and 60 glucose units, 11 and 30 glucose units, 12 and 29 glucose units, 13 and 28 glucose units, 14 and 27 glucose units, 15 and 26 glucose units, 16 and 25 glucose units, 17 and 24 glucose units, 18 and 23 glucose units, 19 and 22 glucose units, 20 and 21 glucose units or any range that the skilled artisan would readily recognize would falls within any of the above ranges. In some examples, the ¹³C-α-limit dextrin has about 12 glucose units, about 13 glucose units, about 14 glucose units, about 15 glucose units, about 16 glucose units, about 17 glucose units, about 18 glucose units, about 19 glucose units, about 20 glucose units, about 21 glucose units, about 22 glucose units, about 23 glucose units, about 24 glucose units, about 25 glucose units, about 26 glucose units, about 27 glucose units, about 28 glucose units, about 29 glucose units, about 30 glucose units, about 31 glucose units, about 32 glucose units, about 33 glucose units, about 34 glucose units, about 35 glucose units, about 36 glucose units, about 37 glucose units, about 38 glucose units, about 39 glucose units, and/or about 40 glucose units. The above ranges represent exemplary ranges, and any specific range that falls between 5 and 200 glucose units is contemplated herein, as an example, although this range is not specifically called out above the range of 17 and 41 glucose units is contemplated to be encompassed in the range of 10 and 60 glucose units. Also, the above recitation of the approximate number of glucose units is exemplary, and any specific number of glucose units that falls within any of the above recited ranges is contemplated herein. For example, a ¹³C-α-limit dextrin comprising between 10 and 60 glucose units, contemplates a ¹³C-α-limit dextrin comprising about 17 glucose units.

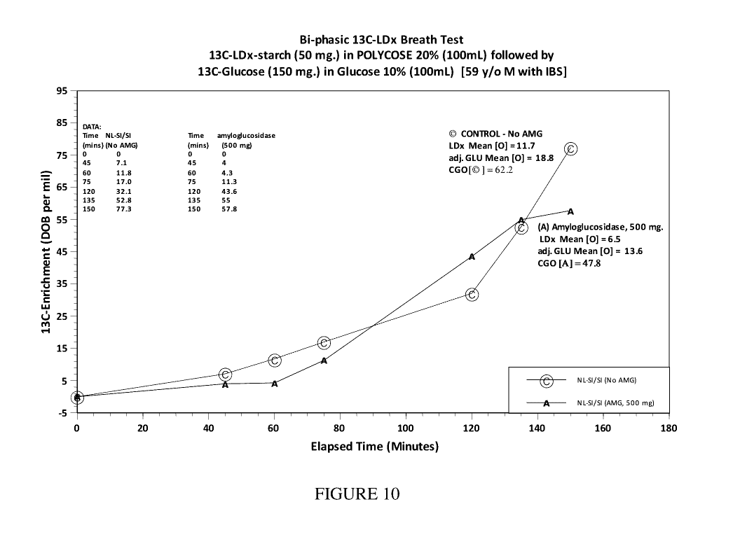

As stated above, the ¹³C-α-limit dextrin is comprised mostly of glucose units having a 1-4 bond linkage, but is also comprised of a limit number of glucose units having a 1-6 bond linkage. The exact n and m values will vary with batch, with the values for m and n being dependent upon botanical origin, method of starch hydrolysis, and duration of starch hydrolysis. In some examples, the value for n is between 5 and 60, 5 and 45, 5 and 40, 5 and 35, 5 and 30, 5 and 25, 5 and 20, 5 and 15, 5 and 10, 5 and 8, 5 and 7, 10 and 60, 15 and 60, 20 and 60, 25 and 60, 30 and 60, 35 and 60, 40 and 60, 45 and 60, 50 and 60, 55 and 60, 11 and 30, 12 and 29, 13 and 28, 14 and 27, 15 and 26, 16 and 25, 17 and 24, 18 and 23, 19 and 22, 20 and 21 or any range that the skilled artisan would readily recognize would falls within any of the above ranges. In some examples, the value for n is about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, and/or about 40. The above ranges represent exemplary ranges, and any specific range that falls between 5 and 200 for n is contemplated herein, as an example, although this range is not specifically called out above the range of 17 and 41 for n is contemplated to be encompassed in the range of between 10 and 60. Also, the above recitation of the approximate value for n is exemplary, and any specific value for n that falls within any of the above recited ranges is contemplated herein. For example, a ¹³C-α-limit dextrin wherein the n is between 10 and 60, contemplates a ¹³C-α-limit dextrin wherein n is about 17. ¹³C-Maltodextrins, because of shorter hydrolysis times, have relatively longer n with an average of 17 glucose units and a reduced value for m (less than 12% 1-6 linkages). See FIG. 10f of reference Nordgaard and Mortensen, “Digestive processes in the human colon.” Nutrition 1995; 11:37-45 in which the contents of this reference are incorporated herein by reference in its entirety.

In some examples, there are reduced 1-6 bond linkages. In other words, the value for m approaches zero in the unbranched amylase fraction which makes up a variable percentage of may botanical species of starches. In other examples, the value of m is between 1 and 10, 1 and 9, 1 and 8, 1 and 7, 1 and 6, 1 and 5, 1 and 5, 1 and 4, and/or 1 and 3. In specific examples, the value of m is 0, 1, 2, 3, 4, 5, 6, 7, 8, 9, or 10. In some examples the sum of m and n equals a value between 5 and 60, 5 and 45, 5 and 40, 5 and 35, 5 and 30, 5 and 25, 5 and 20, 5 and 15, 5 and 10, 5 and 8, 5 and 7, 10 and 60, 15 and 60, 20 and 60, 25 and 60, 30 and 60, 35 and 60, 40 and 60, 45 and 60, 50 and 60, 55 and 60, 11 and 30, 12 and 29, 13 and 28, 14 and 27, 15 and 26, 16 and 25, 17 and 24, 18 and 23, 19 and 22, 20 and 21 or any range that the skilled artisan would readily recognize would falls within any of the above ranges. In some examples, the sum of m and n is about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, and/or about 40. The above ranges represent exemplary ranges, and any specific range that falls between 5 and 200 for sum of m and n is contemplated herein, as an example, although this range is not specifically called out above the range of 17 and 41 for sum of m and n is contemplated to be encompassed in the range of between 10 and 60. Also, the above recitation of the approximate value of the sum for m and n is exemplary, and any specific value for the sum of m and n that falls within any of the above recited ranges is contemplated herein. For example, a ¹³C-α-limit dextrin wherein the sum of m and n is between 10 and 60, contemplates a ¹³C-α-limit dextrin wherein sum of m and n is about 17.

In general when there is a 1-6 linkage present, the ratio of m to n is between 1 to 58 and 1 to 3. In some examples, the ratio of m to n is between, 1 to 58 and 1 to 50, 1 to 58 and 1 to 45, 1 to 58 and 1 to 40, 1 to 58 and 1 to 35, 1 to 58 and 1 to 30, 1 to 58 and 1 to 25, 1 to 58 and 1 to 20, 1 to 58 and 1 to 15, 1 to 58 and 1 to 10, 1 to 58 and 1 to 9, 1 to 58 and 1 to 8, 1 to 58 and 1 to 7, 1 to 58 and 1 to 6, 1 to 58 and 1 to 5, 1 to 58 and 1 to 4, 1 to 58 and 1 to 3, 1 to 58 and 1 to 2, 1 to 58 and 1 to 1. In specific examples, the ratio of m to n is 1 to 58, 1 to 55, 1 to 50, 1 to 45, 1 to 40, 1 to 35, 1 to 30, 1 to 20, 1 to 15, 1 to 10, 1 to 9, 1 to 8, 1 to 7, 1 to 6, 1 to 5, 1 to 4, 1 to 3, 1 to 2 and/or 1 to 1.

In some examples, a solution or a capsule comprises the uniformly ¹³C-α-limit dextrin. In the specific examples of when the ¹³C-α-limit dextrin is contained in a solution, the solution contains 50.00±0.05 g/L glucose equivalents (or multiples thereof), of which only 0.05±0.01 g/L is free glucose. In general, the solution contains between 20 mg/dL and 50 mg/dL glucose equivalents. Another general embodiments, the solution contains less than 0.05 g/L; 0.01 g/L; 0.005 g/L; or, 0.001 g/L free glucose or between 0.05-0.001 g/L; 0.01-0.001 g/L; 0.005-0.001 g/L; 0.05-0.01 g/L; 0.05-0.005; or 0.01-0.005 free glucose. In alternate embodiments, the solution contains less than 50%; 40%; 30%; 25%; 20%; 15%; 10%; 5%; 2%; 1%; 0.1%; 0.05%; 0.01%; 0.005%; 0.001%; 0.0005%; 0.0001%; 0.00005%; 0.00001%; 0.000005%; or 0.000001% free glucose. In some examples, the solution further comprises a 10% Polycose solution or alternate insipient.

In some examples, a solution or a capsule comprises a mixture of uniformly ¹³C-α-limit dextrins. This mixture may comprise one or more types of ¹³C-α-limit dextrin. In specific examples of when the ¹³C-α-limit dextrin is contained in a solution comprising more than one type of ¹³C-α-limit dextrin, the solution contains 50.00±0.05 g/L glucose equivalents (or multiples thereof), of which only 0.05±0.01 g/L is free glucose. In general, the solution comprising more than one type of ¹³C-α-limit dextrin contains between 20 mg/dL and 50 mg/dL glucose equivalents. Another general embodiments, the solution comprising more than one type of ¹³C-α-limit dextrin contains less than 0.05 g/L; 0.01 g/L; 0.005 g/L; or, 0.001 g/L free glucose or between 0.05-0.001 g/L; 0.01-0.001 g/L; 0.005-0.001 g/L; 0.05-0.01 g/L; 0.05-0.005; or 0.01-0.005 free glucose. In alternate embodiments, the solution comprising more than one type of ¹³C-α-limit dextrin contains less than 50%; 40%; 30%; 25%; 20%; 15%; 10%; 5%; 2%; 1%; 0.1%; 0.05%; 0.01%; 0.005%; 0.001%; 0.0005%; 0.0001%; 0.00005%; 0.00001%; 0.000005%; or 0.000001% free glucose. In some examples, the solution containing more than one type of ¹³C-α-limit dextrin further comprises a 10% Polycose solution or alternate insipient.

In some examples, a subject is administered between 0.020 mg and 1000 mg of ¹³C-α-limit dextrin. In some examples, the standard dose is 20 mg for children, 40 mg for small adults, 50 mg for 70 kg to 80 kg adults, and 60 mg for obese adults, but never more than 100 mg when the substrate carbon atom enrichments exceed 95%. In other examples, the subject is administered between 0.05 mg and 1000 mg, 0.1 mg and 1000 mg, 0.5 mg and 1000 mg, 1.0 mg and 1000 mg, 2.5 mg and 1000 mg, 5 mg and 1000 mg, 10 mg and 1000 mg, 25 mg and 1000 mg, 50 mg and 1000 mg, 100 mg and 1000 mg, 250 mg and 1000 mg, 350 mg and 1000 mg, 500 mg and 1000 mg, and/or 750 mg and 1000 mg of ¹³C-α-limit dextrin. In specific examples, the subject is administered 0.025, 0.50, 0.1, 0.5, 2.5, 5, 10, 25, 40, 50, 100, 250, 350, 500, 750 and/or 1000 mg of uniformly ¹³C-α-limit dextrin. In some examples, the ratio of milligrams of ¹³C-α-limit dextrin to milliliters of solution is between 100 to 1 and 0.025 to 1000. In specific examples, the ratio of milligrams of ¹³C-α-limit dextrin to milliliters of solution is between 10 to 1 and 1 to 1000, between 1 to 1 and 1 to 1000, between 1 to 1 and 1 to 100, between 1 to 1 and 1 to 10. In more specific examples, the ratio of milligrams of ¹³C-α-limit dextrin to milliliters of solution is 1 to 1, 1 to 10, 1 to 100, 1 to 1000, 0.025 to 1000, and/or 40 to 100. The above ranges represent exemplary ranges, and any specific range for the ratio of milligrams of ¹³C-α-limit dextrin to milliliters of solution that falls between 100 to 1 and 0.025 to 1000 is contemplated herein, as an example, although this range is not specifically called out above the ratio of 17 to 51 of milligrams of ¹³C-α-limit dextrin to milliliters of solution is contemplated to be encompassed in the range of between 100 to 1 and 0.025 to 1000. Also, the above recitation of the specific ratio of milligrams of ¹³C-α-limit dextrin to milliliters of solution is exemplary, and any specific ratio of milligrams of ¹³C-α-limit dextrin to milliliters of solution that falls within any of the above recited ranges is contemplated herein. For example, a ratio of milligrams of ¹³C-α-limit dextrin to milliliters of solution between 100 to 1 and 0.025 to 1000, contemplates a ratio of milligrams of ¹³C-α-limit dextrin to milliliters of solution of 13 to 63.

The ¹³C-α-limit dextrins disclosed herein are susceptible to brush border hydrolysis. The pancreatic enzymes in the gut could not have a further effect on the substrate and all further hydrolysis in the small bowel is a measure of mucosal brush border disaccharidase activity.

¹³C-α-Limit Dextrin Digestion Breath Test

As stated above, the final step in absorption is for brush border enzymes to convert carbohydrates into single glucose, galactose or fructose units that the body can absorb. There are enzymes (disaccharidases) that convert disaccharides and short chain starch molecules, called α-limit dextrins, into monosaccharide units. In addition to glucoamylase, maltase-glucoamylase plays a part in the last stages of starch digestion, specifically, maltase-glucoamylase is responsible for the hydrolysis of α-limit dextrin. Some dyspeptic children have an intestinal glucoamylase deficiency; others have heterozygous conditions and it appears that others have transient or acquired deficiency of maltase-glucoamylase with or without sucrase-isomaltase deficiency. Some dyspeptic children have a complete congenital intestinal glucoamylase deficiency; others have heterozygous conditions and it appears that others have transient or acquired deficiency of maltase-glucoamylase with or without sucrase-isomaltase deficiency. As a particular example, a non-invasive, stable isotope labeled ¹³C-α-limit dextrin breath test which is used to detect specifically maltase-glucoamylase deficiency.

For example, the ¹³C-α-limit dextrin breath test may be used to measure glucoamylase deficiency and assess the outcome of therapy aimed at improving starch digestion. Under normal conditions, the glucose derived from the enzyme-digested ¹³C-α-limit dextrin is metabolized (oxidized) and appears in the breath as label ¹³C-carbon dioxide which can be measured using a desktop instrument. Conversely, if the ¹³C-α-limit dextrin cannot be digested in the upper small intestine, glucose derived from the starch is “trapped” in the intestine and passes into the colon where intestinal bacteria can metabolize the ¹³C-α-limit dextrin. This process often produces symptoms associated with isomaltase and/or maltase-glucoamylase deficiency and/or ailments resulting from maltase-glucoamylase deficiency. In the event of poor absorption, the isotope label is reduced in breath samples taken during digestion. This principle is also applicable other complex substrates including ¹³C-sucrose (table sugar) and ¹³C-lactose. In some instances, it was found that a comparative reference test using ¹³C-glucose is needed to compensate for individual variations. The results of the two tests (expressed as a calculated coefficient of the two sub-test outcomes, the starch coefficient for glucose metabolism) is indicative of some problems in starch digestion.

The starch coefficient for glucose metabolism is defined as the ratio of ¹³C-carbon dioxide breath enrichment of ¹³C-α-limit dextrin digestion to the ¹³C-carbon dioxide enrichment derived from uniformly-labeled ¹³C-glucose digestion (reference substrate). Glucose absorption is independent of intestinal brush border maltase-glucoamylase (MGAM) activity whereas ¹³C-α-limit dextrin digestion (test substrate) is dependent on MGAM enzyme activity prior to uptake. When ¹³C-α-limit dextrin digestion is impaired, the ratio of the test and reference values decreases (about 80% or greater appears normal). When glucose absorption is abnormal, both ¹³C-α-limit dextrin and ¹³C-glucose oxidations will be reduced.

Breath test substrates are prepared and labeled appropriately for clinical investigations. For example, the ¹³C-α-limit dextrin is diluted in non-isotopically labeled soluble starch (10 gram), suspended as an aqueous beverage and pasteurized. The whole starch may have a ¹³C-atom enrichment greater than 90%. In some examples, the whole starch may have a ¹³C-atom enrichment of at least 10%, of at least 20%, of at least 30%, of at least 40%, of at least 50%, of at least 60%, of at least 70%, of at least 80%, of at least 90%, of at least 92%, of at least 94%, of at least 95%, of at least 97%, of at least 98%, of at least 99% or of 100%. The ¹³C-glucose is derived from the same labeled starch or biochemically by a series of reduction reactions. Breath samples are collected using foil-lined breath collection bags that contain a one-way valve mouthpiece. The breath sample bags, when double sealed, transport easily for analysis or can be analyzed immediately at the bedside.

A two-part carbohydrate oxidation breath test is performed, first using 13C-α-limit dextrin (derived from edible photosynthetic algal starch), in specific embodiments. Breath samples are analyzed using a desktop infrared mass spectrophotometer (UBiT IR300® or POCone®, Otsuka Electronics, Japan). Data are reported for each individual sample as the change in isotopic enrichment as compared to baseline (pre-substrate ingestion) enrichment. Enrichment data (y-axis) is plotted against time of collection (x-axis) and area-under the curve is calculated for each substrate. The primary test outcome is expressed as a ratio of the area-under-the curve result for the 13C-α-limit dextrin substrate data divided by the area under the curve result for the 13C-glucose substrate data. The ideal result should approach unity and impaired absorption is associated with a decreased ratio, (eg, value of 0.80 or less). In some examples, an impaired absorption ration has a value of 0.80 or less, 0.75 or less, 0.70 or less, 0.65 of less, 0.60 or less, 0.55 or less, 0.50 or less, 0.45 or less, 0.40 or less, 0.35 or less, 0.30 or less, 0.25 or less, 0.20 or less, 0.15 or less, 0.10 or less, 0.05 or less and/or 0.025 or less. Subsequently, a uniformly labeled 13C-glucose is used as the substrate. Patients provide 2 to 9 breath samples for isotopic enrichment analysis by inflating (blowing into) a bag (each labeled with subject identifiers, date, time and substrate used). In some cases, a patient or a subject may provide 1 to 100 breath samples, 1 to 90 breath samples, 1 to 80 breath samples, 1 to 70 breath samples, 1 to 60 breath samples, 1 to 50 breath samples, 1 to 40 breath samples, 1 to 30 breath samples, 1 to 25 breath samples, 1 to 20 breath samples, 1 to 18 breath samples, 1 to 15 breath samples, 1 to 13 breath samples, 1 to 11 breath samples, 1 to 10 breath samples, 2 to 10 breath samples, 2 to 9 breath samples, 2 to 8 breath samples, 2 to 6 breath samples, 2 to 5 breath samples, 2 to 3 breath samples, 5 to 15 breath samples, 5 to 13 breath samples, 5 to 11 breath samples, 5 to 7 breath samples, 6 to 12 breath samples, 6 to 10 breath samples 6 to 9 breath samples, 6 to 8 breath samples or breath samples that fall within any of the aforementioned ranges. The substrate (20 mg to 100 mg) is dissolved in a 100 mL 10% solution of edible soluble glucose polymer (Polycose®) and is ingested after collection of the first breath sample (reference-baseline sample, 2 liter) using a foil-lined bag fitted-up with a one-way valve mouthpiece and a double-sealing cap. Subsequent timed test breath samples are collected (250 mL each) at 1, 2, 3, 4, 5, 10, 12, 15, 20, 30, 45, 60 or 120 minute intervals for up to 180 minutes or 3 hours. For example, breath samples may be collected at 10, 20, 30, 40, 45, 50, 60, 70, 75, 80, 90, 100 and/or 120 minutes. The following day, the breath test is repeated in similar fashion, except that the glucose substrate is substituted with ¹³C-α-limit dextrin (10 mg) (derived from edible photosynthetic algae). Breath samples are analyzed using a desktop infrared mass spectrophotometer (UBiT IR300® or POCone®, Otsuka Electronics, Japan). Data are reported for each individual sample as the change in isotopic enrichment as compared to baseline (pre-substrate ingestion) enrichment. Enrichment data (y-axis) is plotted against time of collection (x-axis) and area-under the curve is calculated for each substrate. The primary test outcome is expressed as a ratio of the area-under-the curve result for the ¹³C-α-limit dextrin substrate data divided by the area under the curve result for the ¹³C-glucose substrate data. The ideal result should approach unity and impaired absorption is associated with a decreased ratio, (eg, value of 0.80 or less). In some examples, an impaired absorption ration has a value of 0.80 or less, 0.75 or less, 0.70 or less, 0.65 of less, 0.60 or less, 0.55 or less, 0.50 or less, 0.45 or less, 0.40 or less, 0.35 or less, 0.30 or less, 0.25 or less, 0.20 or less, 0.15 or less, 0.10 or less, 0.05 or less and/or 0.025 or less.

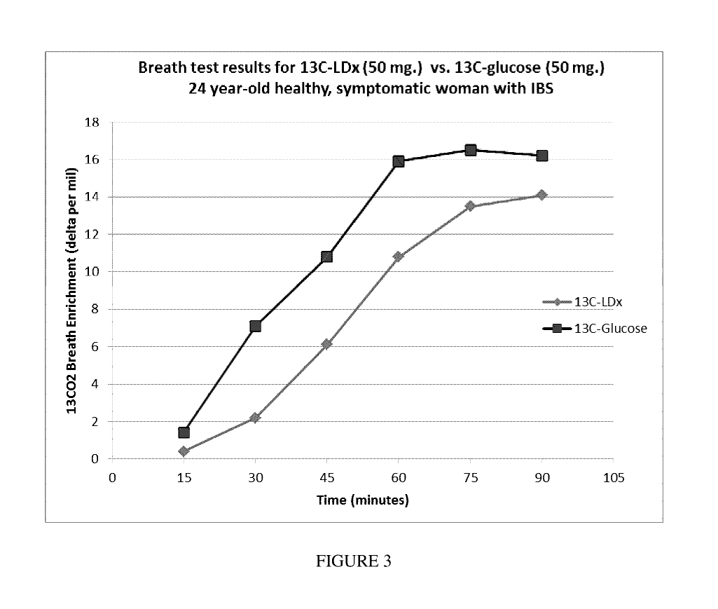

FIG. 1 is a graph that depicts ¹³CO₂ breath enrichment, express as CGO, for eleven healthy control subjects. All subjects reported that they repeatedly ingested large quantities of starch, such as pasta and cereals, without any difficulties or gastrointestinal disturbances, whatsoever. From these data, the best representative breath sampling time-points were determined to be forty-five minutes, sixty minutes and seventy-five minutes and these data established a lower-limit diagnostic cut-off value for the ¹³C-LDx coefficient of glucose oxidation to be 70 percent based upon the margin of errors observed among the resultant mean oxidation data values. Data demonstrate test concept feasibility of using three time points (45 minutes elapsed time, 60 minutes elapsed time and 75 minutes elapsed time). A sample obtained at 90 minutes elapsed time added no additional information. The test substrate is a ¹³C isotopically-labeled LDx substrate, the comparative reference substrate is ¹³C isotopically-labeled glucose and the test principle is illustrated below when two subjects underwent serial ¹³C isotopically-labeled LDx substrate and ¹³C isotopically-labeled glucose breath testing, over two separate days. Two additional healthy subjects were age, gender and BMI matched (aged 24 years, female and BMI=19), whereas the normal subject on depicted on the left was completely asymptomatic and reported tolerance to ingestion of large quantities of dietary starches without any adverse reactions, but the subject depicted on the right had a long history of severe dietary intolerance to ingestion of medium and large quantities of dietary starches with symptoms typical of classic IBS. The data on the left are typical and show normal digestion and oxidation of 13C isotopically-labeled LDx substrate in the asymptomatic subject and no significant difference between LDx digestion and oxidation as compare to reference glucose oxidation. The data on the right are typical and show impaired digestion and oxidation of 13C isotopically-labeled LDx substrate in the symptomatic subject with IBS (67% of predicted 13C-LDx digestive capacity, abnormal by the standard established in this embodiment). FIG. 3 is a patient with symptoms strongly compatible with irritable bowel syndrome, type D and dietary starch intolerance.

Complete starch digestion, or more precisely alpha limit dextrin hydrolysis by mucosal bound enzyme, malto-glucoamylase and sucrose-isomaltase, result in production of glucose that is directly absorbed by the small intestine. The absorbed glucose is further metabolized, and ultimately, results in the production of carbon dioxide which is exhaled and can be detected in the breath of a subject. A starch or more specifically, an α-limit dextrin labeled with the stable isotope (¹³C) or radioactive isotope (¹⁴C) carbon atoms is administered to a subject. The ¹³C-α-limit dextrin is metabolized similarly, which results in isotopically labeled carbon dioxide that is exhaled by the subject and detectable in the breath of the subject. The labeled glucose which comprises the stable isotope (¹³C) or radioactive isotope (¹⁴C) carbon atoms is administered to the subject. The labeled glucose is metabolized which results in isotopically labeled carbon dioxide that is exhaled by the subject and can be detected in the breath of the subject and represents the metabolic fate of all starch or α-limit dextrin molecules that undergo hydrolysis. The ¹³CO₂ measurements resulting from the labeled glucose are compared mathematically as a ratio to the ¹³CO₂ measurement resulting from the hydrolysis of the ¹³C-α-limit dextrin as measured in the timed breath samples (individual reference measurement). Likewise, breath samples, collected before ingestion of the labeled substrate (α-limit dextrin, starch or disaccharides) and periodically after ingestion of substrate can be analyzed using mass spectrometry or gas isotope ratio mass spectrometry (¹³C) or scintillation counting (¹⁴C) to determine the enrichment of isotope derived from test substrates. The ratio of carbon dioxide isotope enrichment derived from starch or α-limit dextrin divided by the isotope enrichment derived from glucose is diagnostic of intestinal glucoamylase, maltose-glucoamylase or disaccharidase deficiency, and in some cases, the absence of intestinal glucoamylase, maltose-glucoamylase or disaccharidase. The deficiency or absence of intestinal glucoamylase, maltose-glucoamylase or disaccharidase appear to be a cause of symptoms similar to those of irritable bowel syndrome or functional bowel syndrome or other inflammatory bowel conditions.

### ¹³C-α-Limit Dextrin Digestion Breath Test Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Clinical Procedure and Technique

A two-part carbohydrate oxidation breath test is performed, first using uniformly labeled ¹³C-glucose as the substrate. Patients provide up to 8 breath samples for isotopic enrichment analysis by inflating (blowing into) a bag (each labeled with subject identifiers, date, time and substrate used). The substrate (50 mg) is dissolved in a 100 mL 20% solution of edible soluble starch (Polycose®) and is ingested after collection of the first breath sample (reference-baseline sample, 2 liter) using a foil-lined bag fitted-up with a one-way valve mouthpiece and a double-sealing cap. Subsequent timed test breath samples are collected (250 mL each) at 30, 45, 60, 75, 90, 105 and 120 minute intervals. The following day or 24 hour period, the breath test is repeated in similar fashion, except that the glucose substrate is substituted with uniformly labeled ¹³C-starch (10 mg). In some examples, the uniformly labeled ¹³C-starch is derived from edible photosynthetic algae. Breath samples are analyzed using a desktop infrared mass spectrophotometer (such as POCone or UBiT IR300, Otsuka Electronics, Japan), though conventional mass spectrometry may be used. Data are reported for each individual sample as the change in isotopic enrichment as compared to baseline (pre-substrate ingestion) enrichment. Enrichment data (y-axis) is plotted against time of collection (x-axis) and area-under the curve is calculated for each substrate. The primary test outcome is expressed as a ratio of the area-under-the-curve result for the ¹³C-starch substrate data divided by the area-under the curve result for the ¹³C-glucose substrate data. The ideal result should approach unity and impaired absorption is associated with a decreased ratio. In some examples, an impaired absorption ratio has a value of 0.80 or less, 0.75 or less, 0.70 or less, 0.65 of less, 0.60 or less, 0.55 or less, 0.50 or less, 0.45 or less, 0.40 or less, 0.35 or less, 0.30 or less, 0.25 or less, 0.20 or less, 0.15 or less, 0.10 or less, 0.05 or less and/or 0.025 or less.

### Example 2

### Assessment of Starch Digestion Capacity

An easy and functional assessment of downstream disaccharide digestion was needed to determine the short-term response to enzyme dietary supplementation and to signal when mucosal function is at fault. Ethical issues and costs make non-invasive BTs after feeding stable-isotope labeled (non-radioactive) carbohydrates an attractive alternative or adjunct to duodenal biopsies for enzyme assays at EGD procedures. A panel of non-invasive breath tests were developed using non-radioactive ¹³C-starch, ¹³C-partially hydrolyzed starch (¹³C-α-LDx), ¹³C-sucrose and ¹³C-glucose substrates that appear promising for clinically measuring deficient starch or sugar digestion at the mucosal level. The digested and absorbed glucose from the ¹³C-starch substrate is oxidized and appears in the breath and is compared to a paired BT of absorbed ¹³C-glucose with the same ¹³C-enrichment. Efficient crude ¹³C-starch digestion and oxidation closely (normally) parallels the glucose reference to a degree of approximately greater than 50% and ¹³C-α-LDx oxidation exceeds 70% per unit time.

A 7-month old female child (JC) presented with chronic starch-induced diarrhea. Duodenal biopsy revealed the following disaccharidase activities: maltase 87 U/gp, sucrase 41 U/gp and normal histology (no inflammatory conditions). The child was compared with 10 controls (dyspeptic children with normal enzyme activities) and 10 CSID controls: (5F: 5M, 11 month old-15 year old). Children with low sucrase activity on duodenal biopsy (<25 U/gp) had BTs were conducted using uniformly-labeled ¹³C-substrates (20 mg each, Isotec, Miamisburg, Ohio) administered in 10 gm Polycose® solution (10%) after an overnight fast. ¹³CO₂ BT (q15 min x 9 samples) were assayed by infrared spectrophotometer (POCone®, Otsuka Electronics, Tokyo, Japan). CGO was calculated for starch ¹³CO₂ enrichment.

TABLE 1
Biopsy Enzyme Activities

| | Maltase | Sucrase | Lactase | Palatinase |
|---|---|---|---|---|
| Population | u/pg | u/pg | u/pg | u/pg |
| JC | 87 | 41 | 44 | 6.8 |
| Normal controls * | 115-268 | 35-96 | 8.5-52 | 7-17 |
| CSID controls * | 0-94 | 0-6 | 23-126 | 0-7 |

The lower normal limit of total maltase activity is 89 u/pg. In JC the maltase was 87 u/pg reflecting persistent SI, but a low total maltase activity. The intermediate maltase activity with normal sucrase (>26 u/pg) led to suspicion of primary MGAM deficiency. In CSID, the mean maltase activity is 44 u/pg reflecting remaining MGAM.

Breath Tests: Glucose BT oxidation outcome value was used as the standard denominator to normalize the percentage of glucose oxidized from starch. The normal control group was analyzed to define a lower limit for the Starch/Glucose ratio. The lowest starch to glucose percentage (% CGO) from normal controls was 17% with a mean of 42%. JC had clinical starch intolerance with % CGO from starch of 6%. Three of 10 CSID's patients had % CGO lower than 17%.

JC is an infant who presented with the clinical picture of starch intolerance. This child had normal histology and a suspiciously low biopsy maltase activity. All ten CSID control patient enzyme levels of sucrase fell below the 10th %, in a range from 0 to 6.5 U/gp, and palatinase (isomaltase) had levels from 0 to 4.9 U/gp; one CSID patient had a normal isomaltase activity (7 U/gp). In CSID control patients, maltase activity varied from 0 to 94 U/gp, suggesting that some patients had both SI and MGAM deficiency. The normal controls all had disaccharidase activities >10%, with the lowest maltase of 115, sucrase of 35 and palatinase of 7 U/gp.

The suspicion of a MGAM basis for maltase reduction in JC was evaluated with a breath test using ¹³C-starch as substrate. In previous studies, it was found that ¹³C-sucrose BTs from 10 CSID patients with biopsy-proven sucrase deficiency and normal histology (Youssef et al., 2006). The ¹³C-starch BT was also done in both the biopsy-proven CSID patients and normal controls. The ¹³C-starch BT of the propositus was much lower than any normal control or CSID control and appeared to confirm isomaltase and MGAM deficiency. It was concluded that the ¹³C-Starch BT provided a non-invasive method for confirmation of poor starch digestion in patients with MGAM insufficiency on biopsy and should be a predictive indicator.

### Example 3

### Normal Limits of ¹³C-Alpha-Limit Dextrin Breath Test

Salivary and pancreatic α-amylases both partially hydrolyze starch by cleaving only internal α-1, 4 glycosyl linkages to three basic types of molecules: maltose (two glucose units with one bond), maltotriose (three glucose units with two bonds), and α-LDx (short glucose chains consisting of α-1, 4 glycosyl linkages with a limited number of residual branching α-1, 6 glycosyl linkages). The length of the α-LDx chain may be longer if the reaction is not allowed to go to completion in vitro. α-LDx are so named because they are digested to the limit to which α-amylase can digest starch. These “limited” products of digestion by amylase are oligosaccharides and disaccharides that are then subsequently hydrolyzed to glucose at the mucosal surface of the small intestine. The benefit of using α-LDx is the elimination of the confounding variable of pancreatic alpha amylase activity in the digestion of complex luminal starch. With the α-LDx product of predigested starch as the indicator substrate, the rate of digestion was theoretically unaffected by individual variations in pancreatic function and became specific to brush border disaccharidase hydrolysis capacity. ¹³C-α-LDx is prepared under GLP conditions in a research kitchen, using a standard protocol, from edible universally stable-isotope labeled whole algal starch (97-99% APE Carbon-13, Isotec, Miamisburg, Ohio; GMP grade).

All subjects in the study performed the ¹³C-glucose and ¹³C-α-LDx BT series. In the first BT series, 40 milligrams of ¹³C-glucose substrate was administered and serial breath measurements were made. On a separate day following dissipation of residual, total body ¹³CO₂ from each subject, a second breath series was done using 40 milligrams of the ¹³C-α-LDx substrate. Subjects were 11 healthy adults aged between 18 and 30 years that consumed a daily regular diet that included starch and 3 adult patients with IBS symptoms. The subjects underwent paired, timed BTs with the administration of ¹³C-glucose and ¹³C-α-LDx, respectively.

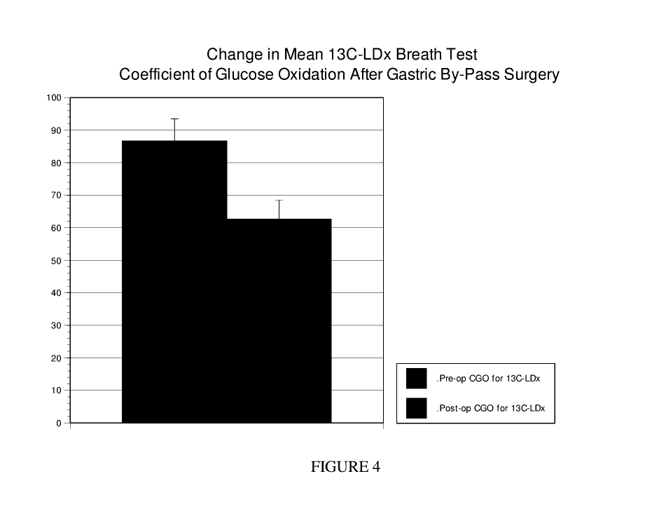

The dose of ¹³C-glucose (Isotec) or ¹³C-α-limit dextrin was equal to 40 milligrams, though the ¹³C-α-LDx contained approximately 30% residual buffer salts. The rate of ¹³C-α-limit dextrin hydrolysis was indirectly measured by the detection of ¹³CO₂ breath sample enrichment over comparative baseline values after the ingestion of ¹³C-glucose and ¹³C-α-limit dextrin. In order to assess the enzymatic capacity of maltase, glucoamylase and sucrase isomaltase, the enrichment values of ¹³C-α-limit dextrin were divided by the ¹³C-glucose values at each time point between 60 and 90 minutes. The Coefficient of Glucose Oxidation (CGO) represents the rate of α-LDx hydrolysis was compared to the rate of glucose absorption. FIG. 4 shows 11 normal asymptomatic control subjects and their ¹³C-breath test starch oxidation data. Also, as shown in FIG. 4 the mean CGO is 75%+/19.

The time frame between 60 and 90 minutes was chosen as reference frame because the coefficient of variance for the control cohort was lowest, representing the best range for predictability of results. This period was also chosen to allow enough time for the substrate to enter the small intestine. After 120 minutes, the data became unpredictable due to substrate depletion and possible colonic interference of distal bacterial fermentation. The mean value of the CGO determined for the eleven control subjects was 74.8 percent. The ¹³C-α-LDx CGO was greater than 50% of glucose in every control subject.

### Example 4

### Irritable Bowel Syndrome (IBS)

In order to show possible ¹³C-α-LDx hydrolysis insufficiency in adult subjects with the diarrhea subtype of irritable bowel syndrome (IBS), ¹³C-α-LDx CGO data for IBS-D subjects and the control data were compared. In two IBS-D subjects (not shown), the data showed decreased early-phase oxidation compared to controls. The mean CGO between minutes 60 and 90 for IBS-D Subject JV was 48% and the mean CGO between 60 and 90 minutes for IBS-D Subject CRC was also 48% of mean control data. One other IBS-Mixed subject with symptoms suggestive of dysmotilty problems (TBS, not shown) had elevated breath hydrogen measurements that suggested an alternative diagnosis of small bowel bacterial overgrowth. This subject's starch CGO was 94%, moderately greater than the control cohorts' data, and his course variably improved with antibiotics.

### Example 5

### Test Article Adjustments

Assays were performed on various over-the-counter nutritional enzyme supplements to independently determine AMG activity. Wide variations were found. Specifically it was found that the Source Natural brand, the brand originally used in the human breath test experiments, had a relatively limited hydrolytic capacity. After an extensive nationwide search, it was found that pure, food grade AMG is available, but is not labeled nor packaged for sale in the USA. However, one approved brand was located which was reported to have a high AMG activity as a component of its recipe (Polyenzyme Plus by Nutriteck (PEP-N), Ultrabiologics, Montreal Canada, US FDA Food Permit #1295276278, http://www.nutriteck.com/polyenzyma.html). In vitro assay results showed excellent glucose release capability from target maltose substrate (0.4 and 0.8 mg/mL maltose substrate (50 mM) at 10 and 30 minutes, respectively). This supplement was more than 10 times more potent that the Source Naturals brand.

Subsequently, a patient with symptoms of abdominal discomfort, difficult bowel movements and excessive flatulence was studied. He was studied twice using the standard ¹³C-α-LDx BT protocol. A test was done without enzyme supplementation and another was done the next day with a single, 1 gram serving of PEP-N added to the BT substrate immediately before ingestion. The results revealed that enzyme supplementation increased ¹³C-α-limit dextrins hydrolysis over 90 minutes. The assumption was made that he had inherently normal digestive capabilities based on absence of dietary intolerances, so the expectations for significantly “improved” digestion over his normal baseline were low, yet a 16% rate increase in oxidation was observed after 60 minutes. This data affirms the concept that the new supplement is better that the original choice. It is expected that children with impaired digestive capacity, secondary to disaccharidase insufficiency, will respond better to a pure form or higher dose of amyloglucosidase. FIG. 10 demonstrates the effect of 500 milligrams amyloglucosidase upon the biphasic ¹³C-α-LDx breath test. This patient had relatively normal digestive capacity and the biphasic test enzyme only marginally affected the rate of digestion. Results are consistent with the expected physiological response to intragastric digestion and the associated down regulation of gastric emptying (pyloro-duodenal osmotic receptor response to osmotic pressure (Barker et al., 1974, PMID: 4822585). Results indicate that free glucose production in the gastric lumen decreased gastric empyting, thereby reducing appearance of 13C-carbon dioxide in the breath, as represented by the decrease in the LDx-starch coefficient of glucose oxidation (CGO) obtained with concurrent amyloglucosidase enzyme administration, 62.2 as compared with control sample CGO 47.8. It is hypothesized that the decrease rate of substrate delivery to the small bowel should equate to overall increased digestion and absorption and decreased symptoms that could be attributed to malabsorption and distal fermentation in the colon. The breath test outcome is silent with regard to efficacy for symptom relief.

### Example 6

### Oral Alpha-Glucosidase Replacement Therapy

In general, to determine if children with FGID and α-glucosidase insufficiency respond to treatment with oral α-glucosidase replacement therapy one must identify symptomatic children, assess them for α-glucosidase insufficiency by using standard biopsy assays, standardized questionnaires and innovative stable isotope-tracing techniques, provide replacement oral enzyme therapy, assess response to replacement therapy and draw conclusions about the impact the assessment and treatment has upon the large population of children that suffer from abdominal pain-related functional gastrointestinal disease (AP-FGID).

A double blind, placebo-controlled study of amyloglucsosidase supplementation to assess improvement of starch and sucrose digestion. Given a frequency of 21-28% glucoamylase insufficiency, in some instances, 350 patients may be screened to identify a qualified test population. Following endoscopy, when research biopsies may be obtained in addition to clinical samples, subjects may undergo ¹³C-α-LDx (starch oxidation) and ¹³C-sucrose (sucrose oxidation) BTs to assess the correlation between endoscopically obtained mucosal enzyme activity biopsy results with breath test results. Research biopsies may be obtained at the time of screening endoscopy in order to fractionate disaccharidase activity (MGAM and SI) by immunoprecipitation. Subsequently, the enrolled subjects may receive oral replacement disaccharidase therapy or placebo under one experimental protocol. Outcome measures include symptom scores, ¹³C-labeled substrate breath test scores expressed as CGO and safety parameters. Research biopsies may consist of 3-4 samples obtained from the mid-distal duodenum. In some instances, all samples may be stored in RNAase-free vials and snap-frozen in liquid nitrogen. In some examples, the samples may be analyzed for total MDase, LDxase, fractional MGAM activity and SI activity.

In specific examples, a 8 mL blood sample may be obtained, and white cells (buffy-coat) may be stored from subjects and available family members for future use. Also, samples may include DNA extraction and sequencing of the MGAM and SI genes to look for possible new mutations.

### Example 7

### Study Population

In some examples, the study population may include symptomatic children diagnosed with IBS (H2b) or FAP (H2d) of sufficient severity as to interrupt normal daily activities or performance, occurring over a period of 3 months, aged 5-15 years-old, presenting for clinically indicated upper-gastrointestinal endoscopy to rule out serious pathology. In some examples, subjects are considered until an indication for upper-gastrointestinal endoscopy has been established. In particular examples, anthropometrics and a detailed diet survey may be performed on each child enrolled.

Examples, of inclusion criteria include informed consent with agreement for storage of identifiable research biopsy samples, a clinical diagnosis of FGID, availability for study participation (access to TCH-GI), the absence of a specific putative diagnosis (lactose intolerance), generally stable medical and psychosocial condition, and not previously known to have Helicobacter pylori infection. In some examples, the clinical plan must independently include clinically indicated upper-gastrointestinal endoscopy. In some instances, the subject is considered to be at “low-risk” for complications of endoscopy.

Examples of exclusion criteria include alarm features, as described by Rasquin et al. (Rasquin-Weber et al., 1999), cognitive impairments that would preclude acquisition of symptom data, active or sub-acute Helicobacter pylori infection (<180 days since cure), diabetes mellitus, infectious enteropathy, gastroduodenal endoscopy that reveals a specific diagnosis such as inflammatory bowel disease, lactose intolerance, celiac disease, NSAID-induced pathology or gross peptic ulcer disease, known allergy to any component of the breath test procedure, allergy (or suspected allergy) to nutritional supplements containing amyloglucosidase, history of atopic diseases, recent abdominal surgery, short-gut syndrome, asthma, chronic bronchitis, obstructive sleep apnea, obesity, clinical renal disease, liver disease, chronic medication use including psychotropic medications, methylphenidate, NSAIDs, antibiotics within 30 days of study, antihypertensive, anti-secretory agents (except that H2 antagonists will be permitted, but not PPIs), anti-seizure medications, motility agents, history of intolerance or allergy to nutritional supplements, and any other condition that, in the opinion of the investigators, could place the subject beyond minimal risk or compromise the safety of the study in any way.

### Example 8

### ¹³C-α-Limit Dextrin Breath Tests and Mucosal MGAM Insufficiency

To confirm the relationship between symptoms scores, ¹³C-α-LDx BTs and mucosal MGAM insufficiency one must examine the effect of oral supplementation with fungal amyloglucosidase on both symptoms and breath test results as surrogate markers for complete digestibility.

For example, one may compare the response to AMG supplementation in 44 study subjects with disaccharidase insufficiency (MGAM<80 U/g) with 44 age/gender-matched controls. Subjects may be randomly receive placebo or test article (AMG supplementation). Demographic, symptom and clinical data may be obtained. The primary outcome variable to assess the response to AMG supplementation is improvement in symptom indices. ¹³C-ST BT outcomes may be measured before and during AMG supplementation therapy. Because of ¹³C-α-LDx substrate overlap in the Dahlqvist clinical assays, research biopsy homogenates may undergo immunoprecipitation to fractionate the starch-digesting enzymes into MGAM and SI complexes.

### Example 9

### ¹³C-Alpha-Limit Dextrin (Starch-Derived) Breath Testing (¹³C-Alpha-Limit Dextrin Breath Test)

Subjects may undergo paired testing that consists of ¹³C-glucose testing that is used to establish an internal reference for carbohydrate oxidative capacity. Glucose oxidation is not dependent upon mucosal disaccharidase activity and provides a benchmark reference for ideal ¹³C-α-limit dextrin hydrolysis. ¹³C-glucose is rapidly metabolized and residual stable isotope tracer dissipates within 12 hours. As such, this test is always done first to avoid influencing outcomes of ¹³C-α-LDx BT. The oxidation data (area under the timed curve for ¹³CO₂ breath enrichment) derived from the ¹³C-α-limit dextrin breath test (¹³C-α-limit dextrin hydrolysis) is expressed as a coefficient of ¹³C-glucose oxidation and may be used for intrasubject and inter-subject comparisons. Real-time analyses against each subject's ¹³C-glucose oxidation reference data factors out the need for direct adjustment to the international limestone standard (Pee-Dee Belemite) or differences in inter-subject metabolic rates.

Subjects must be afebrile and not acutely ill on the days of breath testing. Procedure may be executed as follows: obtain baseline isotopic enrichment (pre-substrate ingestion) breath samples for comparison with post-substrate reference samples, ingest 50 mg of ¹³C-α-LDx or ¹³C-glucose in 20% Polycose® solution (100 mL) and note as “zero” time, collect 12 timed, post-substrate ingestion breath samples (10-120 minutes) and determine comparative isotopic enrichment of each sample using infrared isotopic dispersion mass spectrophotometers (POCone®, Otsuka Electronics, Japan). Breath samples may be collected by asking the subjects to periodically inflate foil-lined collection bags. Each bag is fitted-up with a mouthpiece containing a one-way-valve that easily connects to the spectrophotometer for rapid analysis. ¹³CO₂ breath testing of this type is now widely available and multiple backup instruments are available without incurring additional costs.

Raw data may be expressed as delta-over-baseline (units: delta per mil). Collective enrichment results of ¹³C-α-LDx BT and ¹³C-glucose BT are plotted and area-under-the-curve values are determined for each test. The result of the ¹³C-α-LDx BT is ultimately expressed as a coefficient of the ¹³C-glucose oxidation. Breath test threshold values may be established to permit categorical assignment using receiver-operator characteristic and correlative analyses.

### Example 10

### Trial Design

The MGAM-insufficiency may be a limited double blind, randomized parallel study to assess a subjects response to oral supplementation with fungal-derived amyloglucosidase. Symptom indices, baseline BT and biopsy results may be determined prior to dosing of substrate. Subjects may be administered a 1 gm (2 capsules) of test article (200 units of amyloglucosidase or matched placebo) at the beginning of each meal, planning on 3 meals per day for 3 weeks duration. Standard assessments may be done before and after therapy, including medical history, physical exam, height, body weight (BMI) and laboratory studies (CBC, urinalysis, chemistry panel including fasting serum glucose, 2-hr. pp serum glucose, AST/ALT/GGT, alkaline phosphatase, amylase, and uric acid). At the time of secondary ¹³C-α-LDx BT, test article capsules may be consumed with the breath test substrate in lieu of a meal. After 3 weeks, subjects may return to clinical research clinic for repeat follow-up breath testing and clinical assessments. BT and symptom outcomes may be compared and tested for statistical differences. Standardized diets with established starch content may be prescribed. Dietary intake records may be reviewed, collected and analyzed. Subjects (parents) may keep a standardized symptom diary and report any adverse effects immediately as they occur.

Amyloglucosidase (105 U/gm) in gelatin capsules 500 mg each or matched placebo. Dose and Frequency: two capsules by mouth with meals and before the second ¹³C-α-LDx BT for a duration of 3 weeks. Various safety testing parameters may be evaluated such as document absence of salmonella sp., shigella sp., Campylobacter sp., Enterotoxigenic E. coli sp., E. coli 0157 and aflatoxin contamination. Also, a pepsin stability test may be conducted, as well as a dissolution test: In some instances, after Lages and Tannenbaum an in-vitro activity test is performed. Amyloglucosidase (21CFR173.110), also known as carbohydrase 27 C.F.R. 240.1051b is also cited by the Office of Food Additive Safety—Center for Food Safety & Applied Nutrition of the U. S. Food and Drug Administration list of Microbial-Derived Ingredients That Are Used in Foods (21 C.F.R. 123.130). In some examples a subject is given a placebo. In general, the placebo comprises matched gelatin capsules with inert ingredients.

Descriptive statistics may be performed on the data set. All statistical tests may be performed by using StatView software (Abacus, Calif.). Means, SDs, medians and extremes may be calculated for all quantitative parameters. The primary statistical test may be a test of proportional clinical and metabolic response to therapy (Fisher Exact Tests) applied to breath-test outcomes and stratified symptom scores. Spearman's Rank-order Correlation Coefficient P (rho) will be applied to scores. A P value of less than 0.05 may be taken as statistically significant. Regression analysis may be done to compare enzyme activity obtained from biopsy assays with BT outcomes. Receive-operator-characteristic may be used to determine diagnostic threshold values for breath test enrichment values. BT outcomes may also be compared with symptom scores and enzyme assay results.

In some examples, it may be necessary to fractionate α-glucosidase biopsy activities. Both MGAM and SI each have two active sites which hydrolyze maltosaccharides with different specificities and rates. Comparison of the measured shared mucosal activities with the ¹³C-α-LDx BT results is not a simple task as with sucrase because of the overlapping activities; a first step toward specificity will be to immunopurify the MGAM by immunoprecipitation of SI activity. Previously, this method has been validated (Sauer et al., 2000).

### Biphasic ¹³C-Sucrose/13C-Glucose Breath Test Validation Experiment

It was observed that cachectic patients, with cancer and FBD-like symptoms, have markedly diminished ¹³C-carbohydrate oligomer (e.g. ¹³C-LDx)/glucose breath test oxidation scores that worsen with chemotherapy (unpublished results). A new biphasic breath test, described herein, is useful for the evaluation of gastrointestinal mucosal enzyme (disaccharidases such as sucrase, maltase, glucoamylase, isomaltase) insufficiency in various clinical conditions, including functional bowel syndrome and iatrogenic mucosal enzyme loss secondary to chemotherapy or infection. The biphasic breath test was performed using ¹³C-sucrose as the target substrate using subjects that have a genetic aberration specific to the SUCRASE domain of the sucrase-isomaltase gene (3q25.2-q26.2).

It is unlikely that the biphasic ¹³C-carbohydrate oligomer (e.g., ¹³C-LDx)/glucose could be validated by direct comparison with mucosal biopsies and sample assays since the sampling could not be assured to be representative of the entire bowel digestive capacity and the approach is cost prohibitive. Furthermore, much of the luminal mucosal digestive surface is beyond the reach of the endoscope and proximal duodenal biopsies are not always representative of distal jejunal biopsy disaccharidase activity. In addition, the numbers of mucosal samples needed to make a comparison are beyond what would be considered safe to obtain from human volunteer subjects. As such, an alternative validation approach was needed to determine if the biphasic ¹³C-carbohydrate oligomer (e.g., ¹³C-LDx)/¹³GBT measures end-stage digestion.

### Example 11

### Biphasic ¹³C-Sucrose/¹³C-Glucose Breath Test Method

Study Population: Four adult, non-diabetic women with specific genetic mutations were recruited as subjects for a sub-study based upon their sucrase-isomaltase (SI) genotype (3q25.2-q26.2 mutation status) and detailed self-reported dietary carbohydrate tolerance. One subject was markedly symptomatic for sucrose and starch ingestion and homozygous for congenital sucrase-isomatase (SI) deficiency, two subjects were heterozygous for congenital sucrase-isomatase deficiency in the sucrase gene domain and moderately symptomatic for sucrose and starch ingestion, and one subjects was carbohydrate tolerant for sucrose and starch ingestion (completely asymptomatic) without any of the known mutations in the sucrase-isomatase gene.

Subject-A was a healthy, asymptomatic 45.9 year-old woman that routinely ingested dietary sucrose without adverse reactions; her SI genotype (WT/WT) revealed no genetic aberrations by the ABI PRISM SNaPshot Assay (Life Technologies, performed by LabCorp test 511570 SI, RTP, North Carolina).

Subject-D was a profoundly symptomatic 46.7 year-old woman that avoided dietary sucrose and required sacrosidase-supplementation with most meals to avoid typical symptoms of maldigestion; her SI genotype revealed homozygous aberrations [c.3218G>A(p.G1073D)]. A recent mucosal biopsy revealed no sucrase activity (only one sample obtained).

Subject-B was a moderately symptomatic 23.3 year-old woman that restricted dietary sucrose to avoid typical symptoms. She was the biologic daughter of Subject-D and her sucrase-isomaltase genotype revealed heterozygous aberrations [c.3218G>A; (p.G1073D)/WT]. She has not required sacrosidase supplementation and manages her sucrose ingestion by dietary restriction.

Subject-C was a moderately symptomatic 43.9 year-old woman with a different SI mutation [c.5234T>G; (p.P1745C)] that also restricted dietary sucrose to avoid typical symptoms and periodically requires sacrosidase supplementation. A sucrase assay of mucosal biopsy performed 13 years earlier demonstrated diminished sucrase activity (25 μm/min/gm protein: exactly at the diagnostic threshold, 5th percentile).

Mucosal sucrase is expressed concomitantly with isomaltase as part of the same gene products; then each component is activated after expression by endopeptidase cleavage. As such, the use of ¹³C-sucrose can be a surrogate for ¹³C-LDx or ¹³C-starch. The key concept is that free glucose does not appear in circulating blood, to any appreciable degree, unless luminal enzyme hydrolysis first occurs to release glucose for absorption.

### Example 12

### Breath Testing after Biphasic ¹³C-Sucrose/¹³C-Glucose Administration

Commercial Mylar® breath collection bags (1.3 L and 0.25 L) fitted with one-way valves (Otsuka Electronics, Tokyo Japan) were used. Mouthpieces were fashioned from drinking straws to facilitate inflation by study participants and establish airtight connections between airway and collection bags in order to reduce room air cross-contamination. Each kit contained 5 bags labeled for timed collections: 1 large bag for baseline collection for repeated reference enrichment comparisons and 4 test sample bags for 60-minute and 75-minute breath collections post-sucrose and post-glucose substrate ingestions (delta-over baseline) as measured oxidation values typically peak between these time points in normal subjects, at tracer dosage levels.

¹³C-tracer isotopes (Isotec, Div. of Sigma Aldrich, Miamisburg, Ohio) and aqueous excipients were prepared and labeled under a clean Class 5 bench hood in 100 mL volumes [MediPak USP sterile water (37-6250), McKesson Medical-Surgical, Richmond, Va.]. Dosed tracers and aqueous excipients composing the substrates were weighed analytically, mixed by combining 25.0 mg uniformly labeled ¹³C-sucrose and 125.0 mg uniformly labeled ¹³C-glucose with 15 grams unlabeled sucrose and 15 grams unlabeled glucose, respectively, as challenge loads. The unlabeled challenge load gram-weight was selected because it approximates a threshold unlikely to induce symptoms or delay gastric emptying in most adults. To avoid confusion, substrates were color-coded using McCormick® food coloring (blue for sucrose, pink for glucose) to match the color-coordinated sample collection bags, and flavored respectively with Adams® natural strawberry and coconut extracts for taste and aroma. All substrates were frozen at −20° C. until needed in Nalgene® polyethylene bottles. Breath testing was performed in the clinic, home or laboratory setting in the early morning hours before 12 p.m. after an overnight fast.

At study initiation, subjects were asked to hold-their-breath for a 10-count and inflate the reference breath sample for isotopic comparison later with timed breath samples; then ¹³C-sucrose substrate solution, with 15% excipient, was ingested (at time 0). At 60 minutes post-¹³C-sucrose ingestion, a breath sample was collected, followed by another breath sample at 75 minutes. Immediately after sample collections related to sucrose ingestion were complete, a comparative ¹³C-glucose substrate solution was ingested (part II of the bi-phasic breath test) to assess inherent rate of metabolism for free glucose. This was used for comparison with part I outcomes (see calculations). The super-dose (125 mg, 5-times the ¹³C-sucrose dose) was intended to dilute (mask) any residual ₁₃CO₂ carry-over effects from the sucrose and to assess inherent body oxidation capacity. As before, breath samples were collected at 60 minutes and 75 minutes post 13C-glucose substrate ingestion and results were adjusted by a factor of 0.2× to account for super-dosing and to compare, pairwise, ¹³C-sucrose oxidation data with ¹³C-glucose data. Eight milliliter blood samples were collect concomitant with each breath sample collection. Post-procedure breath test kits and blood samples were promptly returned to the laboratory for analysis.

Measurement of ¹³CO₂ sample enrichments was performed for each 60-minute and 75-minute breath collection using a ¹³CO₂ mass dispersive infrared spectrometer (POCone®, Otsuka Electronics, Tokyo, Japan). Each sample was analytically compared with the common baseline sample (pre-substrate ingestion sample) and breath test data were expressed as a ratio of the change in ¹³CO₂ enrichment (delta % c) between the baseline sample and the periodically timed, post-substrate ingested samples [delta % c over baseline (DOB) at 60 and 75 minutes (DOB 60′) and (DOB 75′)].

The plasma samples were processed in a closed reaction system where aliquots of plasma (2.00 mL) were incubated (37° C.) overnight with a 2 mL live milk culture of baker's yeast (Red Star, Cedar Rapids, Iowa). Each test tube was fitted to a standard 250 mL Mylar® breath collection bag and each system was primed with 20.0 mL oxygen (100%). After 24-hours elapsed time, the system was purged with 100.0 mL of carrier gas containing 3% unlabeled (¹²C) carbon dioxide. Samples were analyzed for ¹³CO₂ enrichment by comparison with a reference standard using mass dispersive spectrophotometry, adjusted for individual plasma volume (DOB*PV) and results were stratified according to genotype.

Graduated media bottles were identified as optimal. Labeled as 100 mL, but actually hold 150 mL and accept a #3 two-holed rubber stopper. The rubber stopper is fitted up with a male breath bag adaptor with O-rings (nipple connector). The nipple connector is connector to ⅛^{th} inch tubing passed through one hole of the stopper and trimmed flush with the stopper's inner surface. The tubbing was pre-treated with RTV silicon gasket maker goo (Ultra-Blue, #81724) to ensure a gas tight fit. The other hole remained double-sealed with the rubber diaphragm and double-seal using a ½ ml micro-conical centrifuge tube as a secondary, gas-tight stopper.

2.00 mL of serum (of the appropriate time-point) was placed in the reaction bottle with 2 marbles (as mixers) and 5.00 mL of active baker's yeast culture in skim milk and 5% glucose. After flushing the bottle with 30 mL or 100% oxygen, the bottle was sealed with the stopper and a deflated breath collection bag (Otsuka) suitable for use with the POCone infrared spectrophotometer.

The procedure was repeated for all time points (shown below) and in duplicate. The bottles were incubated at 37*C in a water bath for 24-hours (mixed once after 6 hours E.T.). After 24-hours, the microtube was removed and the diaphragm pierced with a blunt needle connected to a tap water supply. The bottle was filled to the brim to displace most gas in the vessel and taking great care not to let water enter the bag. The bag was analyzed for ¹³CO₂ enrichment in usual fashion against a common reference sample and all results were tabulated.

### Example 13

### Calculations

Because of body habitus and age-related variations in carbon dioxide production, the ¹³CO₂ oxidation value for ¹³C-glucose (Part II) was used to adjust for different inter-subject ¹³C-sucrose (Part I) oxidation rates. Oxidized components derived from ¹³C-sucrose (fructose and glucose) were compared to oxidized enrichment values and expressed components derived from the secondary ¹³C-glucose super-dose (⅕×glucose enrichment). After mathematical adjustment for super-dosing of glucose, the enrichment ratio of ¹³CO₂ was attributed to sucrose digestion, absorption and metabolism. (Note: glucose requires no further digestion). All glucose oxidation-related values were universally adjusted by a standard factor (1.12×) to account for minor inherent increased rate in fructose metabolism as compared with glucose (no-isomerization needed) and the 17% proportionally greater atomic weight of sucrose. The ratio of the two ¹³CO₂ enrichments were stated as the Coefficient of Glucose for Sucrose (CGO-S).

### Example 14

### Examples of Results

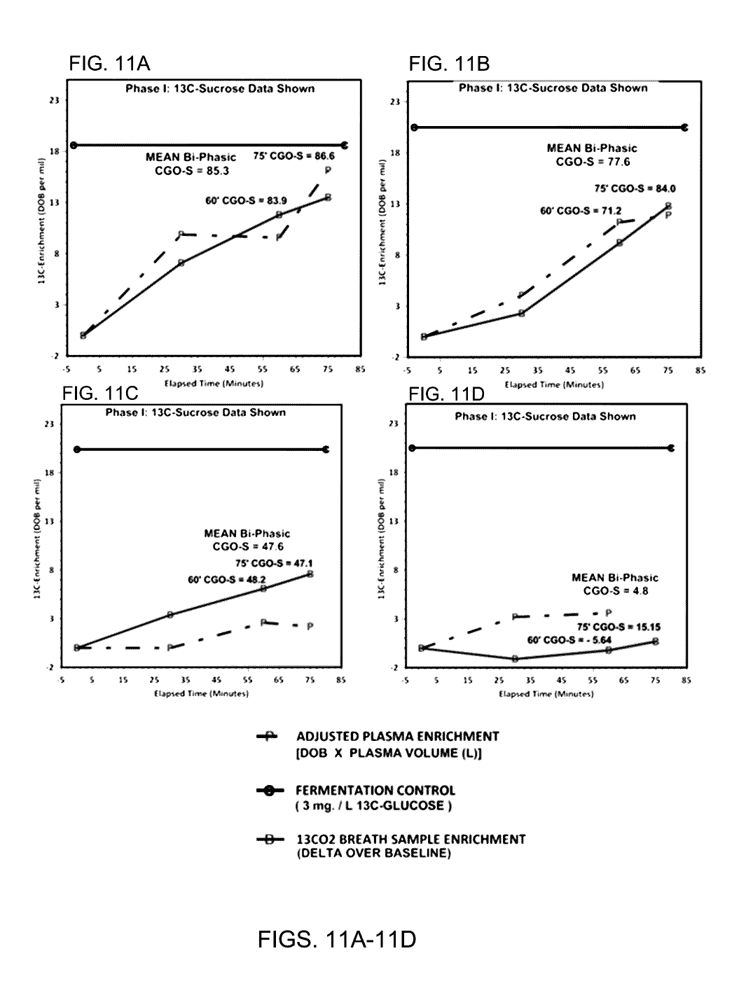

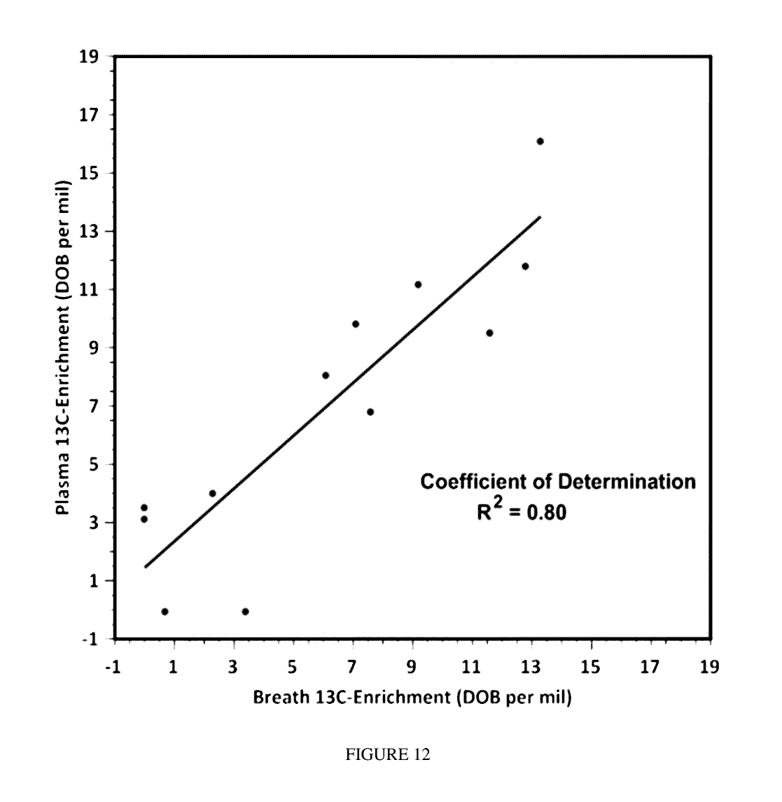

The four genetically SI-characterized subjects undertook the ¹³C-S/GBT with simultaneous breath and blood sampling to validate outcomes. Results are shown in FIG. 11. Panel A (Subject-A: genotype WT/WT) breath enrichments were compared with blood test enrichments and data closely paralleled each other and were markedly higher than the results obtained from Subject D (SI homozygous mutation genotype c.3218G>A); whose results for the sucrase portion of the test approached zero. As predicted the results for the heterozygous subjects (B&C) were moderately depressed compared to Subject-A, and were markedly improved over the results obtained for the SI-affected Subject-D. The mediocre results were consistent with those to be expected for the heterozygous subjects. Collectively, the results obtained the blood assay (fermentation products of digestion) closely correlated (r²=0.80) with ¹³CO₂ breath enrichment (FIG. 12) which indicates successful measurement of end-product metabolism of digested sucrose.

The parallel breath and blood testing for ¹³C-labled products of sucrose digestion in the patients with SI gene mutations, used as a surrogate for assay of random intestinal biopsies, successfully validated use of the ¹³C-carbohydrate oligomer/GBT to screen for carbohydrate maldigestion as a potential cause for maldigestive symptoms. Considering that successful sucrose digestion results in absorption of ¹³C-fructose and ¹³C-glucose, for which ¹³C-fructose is mostly taken up by the liver on first portal circulation pass and that most resultant ¹³C-glucose passes to the systemic circulation for immediate use by vital organs, blood sugar samples were obtained to indirectly assess sucrose digestion independent of mucosal biopsies. It was well known that approximately two-thirds of the glucose and one-quarter of the fructose or half of the total stable isotopic-tracer load was expected to appear in the systemic circulation and to be available as a proportional indicator of normal digestion, since very little free sucrose is passively absorbed by the intestines. Most absorbed fructose is converted to trioses in the liver over time, and trioses are subsequently released into the systemic circulation as lactate for oxidation in extrahepatic tissues or stored as glycogen. Breath sample enrichment is dependent upon further down-stream metabolic activity. Plasma samples were obtained concurrent with timed biphasic ¹³C-sucrose breath test sampling at 30, 60 and 75 minutes after substrate ingestion and assayed. The plasma ¹³C-sugar content was assayed using a novel fermentation assay and gas samples were quantitatively eluted from reaction vessels, similarly analyzed for ¹³CO₂ enrichment and outcomes from plasma and breath samples were compared. The breath samples were analyzed for ¹³CO₂ enrichment by infrared mass dispersive spectrophotometry. As predicted, the plasma content of ¹³C-labeled sugars, mostly ¹³C-glucose, closely paralleled breath-sample enrichment as the appearance of ¹³CO₂ and indicate that the breath test is a valid test of oligosaccharide digestion. Results from ¹³C-LDx experiments using subjects that have a genetic aberration specific to the isomaltase domain of the sucrase-isomaltase gene (3q25.2-q26.2) are forthcoming.

### ¹³C-LDx Breath Test Validation Experiment

The biphasic ¹³C-LDx/¹³GBT that specifically targets mucosal digestive enzymes, described herein, appears useful for the evaluation of gastrointestinal mucosal enzyme insufficiency (disaccharidases including maltase, glucoamylase, isomaltase) in various clinical conditions, including functional bowel disorder syndrome and iatrogenic mucosal enzyme loss secondary to chemotherapy or infection. A validation approach to determine if the biphasic ¹³C-carbohydrate oligomer (e.g., ¹³C-LDx)/¹³GBT measures end-stage digestion is presented.

The validation experiment confirms that ¹³C-LDx/¹³GBT a valid measure of intestinal oligosaccharide digestion. The general approach examines the principle that solely monosaccharides are absorbed into the blood circulation, such that isotopically-labeled tracer oligosaccharides cannot appear directly in plasma, but must undergo complete digestion. The appearance of ¹³C isotopic tracer in the plasma, after oligosaccharide substrate ingestion, indicates the occurrence of digestion. Moreover, the appearance of ¹³C isotopic tracer in the plasma concurrent with the proportional appearance of ¹³C isotopic tracer in the breath, as ¹³C-labeled carbon dioxide, validates the use of the ¹³C-LDx/¹³GBT. The following experiment demonstrates that the breath test distinguishes starch maldigestion.

### Example 15

### ¹³C-LDx Breath Test Validation Methods

Study Population: two non-diabetic women with specific genetic mutations were recruited as subjects under IRB-approved protocol, with documented informed consent, for a sub-study based upon their sucrase-isomaltase (SI) genotype [3q25.2-q26.2 mutation status, ABI PRISM SNaPshot Assay (Life Technologies, performed by LabCorp test 511570 SI, RTP, North Carolina)] and detailed self-reported dietary carbohydrate intolerances, one to starch and one to sucrose (table sugar). One subject (Subject X) was markedly symptomatic for starch and sucrose ingestion and possessed the heterozygous gene mutation in the isomaltase gene domain that caused miscoding for proline for leucine at position 348. The other subject (Subject Z) was moderately symptomatic for sucrose, but relatively starch ingestion tolerant and possessed the heterozygous gene mutation in the sucrase gene domain that caused miscoding for phenylalanine for cysteine at position 1745. Both subjects were otherwise healthy.

It is important to recognize that, in normal health, mucosal sucrase is expressed concomitantly with isomaltase as part of the same gene products; then each component is activated after expression by endopeptidase cleavage. The key concept is that free glucose, derived from oligosaccharide and dextrin hydrolysis, does not appear in circulating blood or become subsequently available for oxidation to ¹³CO₂, to any appreciable degree, unless luminal enzyme hydrolysis occurs first from active isomaltase to release glucose for absorption. Some hydrolysis may occur from a separate gene product that would be expected to maintain some digestive capacity (not zero).

### Example 16

### Description of Study and Breath Testing

Commercial Mylar® breath collection bags (1.3 L and 0.25 L) fitted with one-way valves (Otsuka Electronics, Tokyo Japan) were used. Mouthpieces were fashioned from drinking straws to facilitate inflation by study participants and establish airtight connections between airway and collection bags to reduce room air cross-contamination. Each kit contained five bags labeled for timed collections: 1 large bag for baseline collection for repeated reference enrichment comparisons and 4 test sample bags for 60-minute and 75-minute breath collections post-sucrose and post-glucose substrate ingestions (delta-over-baseline) as measured oxidation values typically peak between these time points in normal subjects, at tracer dosage levels.

¹³C-tracer isotopes (Isotec, Div. of Sigma-Aldrich, Miamisburg, Ohio) and aqueous excipients were prepared and labeled under a clean Class 5 bench hood in 100 mL volumes [MediPak USP sterile water (37-6250), McKesson Medical-Surgical, Richmond, Va.]. Dosed tracers and aqueous excipients composing the substrates were weighed analytically, mixed by combining 50.0 mg uniformly labeled ¹³C-alpha limit dextrin and 150.0 mg uniformly labeled ¹³C-glucose with 15 grams unlabeled glucose polymer (dextrins) solution (100 mL) and 10 grams unlabeled glucose solution (100 mL), respectively, as challenge loads. The unlabeled challenge load gram-weight was selected because it approximates a threshold unlikely to induce symptoms or delay gastric emptying in most adults. To avoid confusion, substrates were color-coded using McCormick® food coloring (blue for ¹³C-alpha limit dextrin, pink for glucose) to match the color-coordinated sample collection bags and flavored respectively with Adams® natural grape and strawberry extracts for taste and aroma. All substrates were frozen at −20° C. until needed in Nalgene® polyethylene bottles. Breath testing was performed in the clinic setting in the early morning hours before 12 p.m. after an overnight fast.

At study initiation, subjects were asked to hold-their-breath for a 10-count and inflated the reference breath sample for isotopic comparison later with timed breath samples; then ¹³C-LDx substrate solution, with 15% excipient, was ingested (at time 0). At 60 minutes post-¹³C-LDx ingestion, a breath sample was collected, followed by another breath sample at 75 minutes. Immediately after sample collections related to sucrose ingestion were complete, a comparative ¹³C-glucose substrate solution was ingested (part II of the bi-phasic breath test) to assess inherent rate of metabolism for free glucose. This was used for comparison with Part I outcomes. The super-dose (150 mg, 3-times the ¹³C-LDx dose) was intended to dilute (mask) any residual ¹³CO₂ carry-over effects from the LDx and to assess the inherent body oxidation capacity of glucose derived from digestion of LDx. As before, breath samples were collected at 60 minutes and 75 minutes post ¹³C-glucose substrate ingestion. Eight-milliliter blood samples were collect concomitant with each breath sample collection. Post-procedure breath test kits and blood samples were promptly returned to the laboratory for analysis.

Measurement of ¹³CO₂ sample enrichments was performed for each 60-minute and 75-minute breath collection using a ¹³CO₂ mass dispersive infrared spectrometer (POCone®, Otsuka Electronics, Tokyo, Japan). Each sample was analytically compared with the common baseline sample (pre-substrate ingestion sample) and breath test data were expressed as a ratio of the change in ¹³CO₂ enrichment (delta % c) between the baseline sample and the periodically timed, post-substrate ingested samples [delta % c over baseline (DOB) at 60 and 75 minutes (DOB 60′) and (DOB 75′)].

The plasma samples were processed in a closed reaction system where aliquots of plasma (2.00 mL) were incubated (37° C.) overnight with a 2 mL live milk culture of baker's yeast (Red Star, Cedar Rapids, Iowa). Each test tube was fitted to a standard 250 mL Mylar® breath collection bag and each system was primed with 20.0 mL oxygen (100%). After 24-hours elapsed time, the system was purged with 100.0 mL of carrier gas containing 3% unlabeled (¹²C) carbon dioxide. Samples were analyzed for ¹³CO₂ enrichment by comparison with a reference standard using mass dispersive spectrophotometry, adjusted for individual plasma volume (DOB*PV) and results were stratified according to genotype.

Graduated media bottles were identified as optimal. Labeled as 100 mL, but actually hold 150 mL and accept a #3 two-holed rubber stopper. The rubber stopper is fitted up with a male breath bag adaptor with O-rings (nipple connector). The nipple connector is connector to ⅛th inch tubing passed through one hole of the stopper and trimmed flush with the stopper's inner surface. The tubbing was pre-treated with RTV silicon gasket maker goo (Ultra-Blue, #81724) to ensure a gas tight fit. The other hole remained double-sealed with the rubber diaphragm and double-seal using a ½ ml micro-conical centrifuge tube as a secondary, gas-tight stopper.

2.00 mL of serum (of the appropriate time-point) was placed in the reaction bottle with 2 marbles (as mixers) and 5.00 mL of active baker's yeast culture in skim milk and 5% glucose. After flushing the bottle with 30 mL of 100% oxygen, the bottle was sealed with the stopper and a deflated breath collection bag (Otsuka) suitable for use with the POCone infrared spectrophotometer.

The procedure was repeated for all time points (shown below) and in duplicate. The bottles were incubated at 37*C in a water bath for 24-hours (mixed once after 6 hours E.T.). After 24-hours, the microtube stopper was removed and the diaphragm pierced with a blunt needle connected to a tap water supply. The bottle was filled to the brim to displace most gas in the vessel and taking great care not to let water enter the bag. The bag was analyzed for ¹³CO₂ enrichment in usual fashion, using the infrared spectrophotometer, and compared with a common reference sample and all results (change of isotopic enrichments) were tabulated.

### Example 17

### Calculations

Because of body habitus and age-related variations in carbon dioxide production, the ¹³CO₂ oxidation value for ¹³C-glucose (Part II) was used to adjust for different inter-subject ¹³C-LDx (Part I) oxidation rates. Oxidized components derived from ¹³C-LDx were compared to oxidized enrichment values and expressed components derived from the secondary ¹³C-glucose super-dose ⅓ x glucose enrichment). After mathematical adjustment for super-dosing of glucose, the enrichment ratio of ¹³CO₂ was attributed to sucrose digestion, absorption and metabolism. (Note: glucose requires no further digestion). The ratio of the two substrate-derived ¹³CO₂ breath test enrichments were stated as the Coefficient of Glucose for LDx (CGO-LDx). All plasma enrichments values were multiplied by 120 mL to account for dilution to eluted gas and divided by 2 to account for the use of 2 mL sample volumes (factor: plasma value=raw enrichment X (120/2).

### Example 18

### ¹³C-LDx Breath Test Validation Results

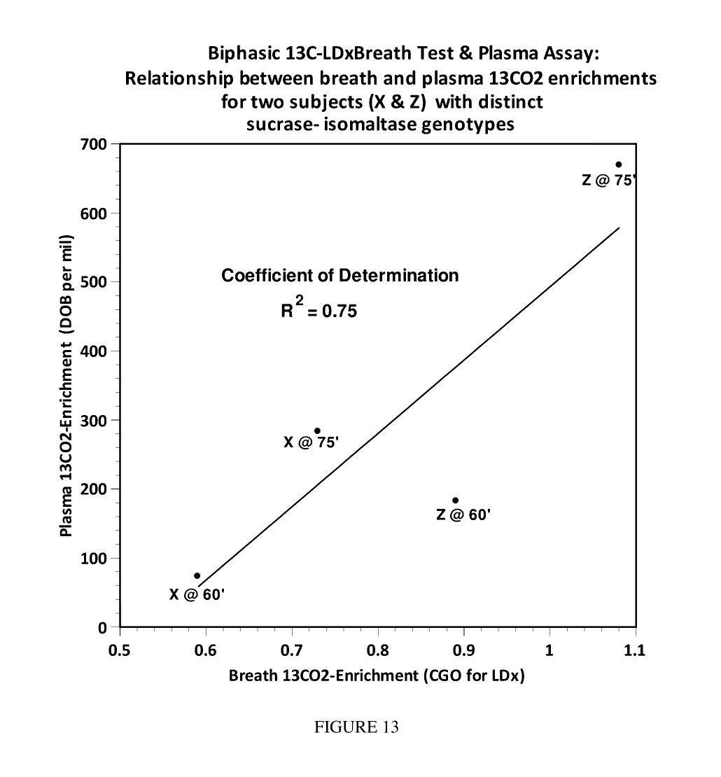

The two SI-characterized subjects undertook the ¹³C-LDx/GBT with simultaneous breath and blood sampling to validate outcomes (Table 1). Results are shown in FIG. 13. Panel A (Subject-X: heterozygous isomaltase mutation) breath enrichments were compared with blood test enrichments and data closely paralleled each other and were markedly lower than the results obtained from Subject Z (heterozygous sucrase mutation); whose results for the ¹³C-LDx/GBT approached unity, as predicted. The results obtained from subject X were consistent with those to be expected for the heterozygous subject with low isomaltase. Collectively, the results obtained the blood assay (fermentation products of digestion) closely correlated (r²=0.75) with ¹³CO₂ breath enrichment (FIG. 13) which indicates the successful measurement of end-product metabolism of digested 13C-LDx.

TABLE 1
Experimental Data obtained for two subjects with sucrase-isomaltase mutations and
faulty starch digestion (Subject X) and faulty sucrose digestion (Subject Z).

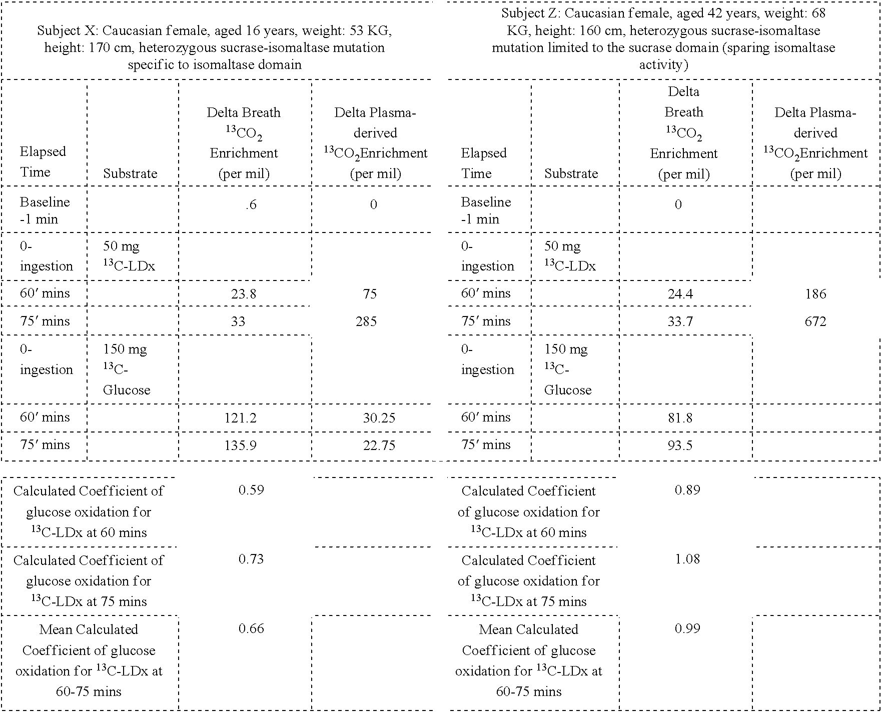

### Example 19

### ¹³C-LDx Breath Test Validation Discussion

The parallel breath and blood testing for ¹³C-labled products of LDx digestion in the two patients with specifically different SI gene mutations, used as a surrogate for assay of random intestinal biopsies, successfully validated use of the ¹³C-¹³C-LDx/GBT to screen for carbohydrate maldigestion as a potential cause for maldigestion symptoms. Considering that successful starch and glucose oligomer digestion results in absorption of ¹³C-glucose, for which most resultant ¹³C-glucose passes to the systemic circulation for immediate use by vital organs to produce ¹³CO₂, blood sugar samples were obtained to indirectly assess LDx digestion independent of mucosal biopsies. It was well known that approximately two-thirds of the derived free glucose was expected to appear in the systemic circulation and to be available as a proportional indicator of digestion. Breath sample enrichment is dependent upon further down-stream metabolic activity. Plasma samples were obtained concurrent with timed biphasic ¹³C-LDx breath test sampling at 60 and 75 minutes after substrate ingestion and assayed. The plasma ¹³C-sugar content was assayed using a novel fermentation assay, and gas samples were quantitatively eluted from reaction vessels, similarly analyzed for ¹³CO₂ enrichment and outcomes from plasma and breath samples were compared. The breath samples were analyzed for ¹³CO₂ enrichment by infrared mass dispersive spectrophotometry. As predicted, the plasma content of ¹³C-labeled sugars, as indicated by the in vitro yeast fermentation product ¹³CO₂, and closely paralleled the in vivo 13CO2breath-sample enrichments. It is concluded that the ¹³C-LDx/glucose breath test is a valid test of mucosal oligosaccharide digestion, independent of alpha-amylase activity.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

All patents and/or publications mentioned in the specification are indicative of the level of those skilled in the art to which the invention pertains. All patents and publications are herein incorporated by reference in their entirety to the same extent as if each individual publication was specifically and individually indicated to be incorporated by reference.
Ao Z, Quezada-Calvillo R, Sim L, Nichols, B L, Rose D R, Sterchi E E, Hamaker B R. Evidence of native starch degradation with human small intestinal maltase-glucoamylase (recombinant). FEBS Letters 2007; 58:2381-2388. PMID: 17485087.
Auricchio S, Ciccimarra F, Jos J, Rey F, Moauro L, Rey J. Studies on intestinal digestion of starch in children with congenital deficiency of sucrase and isomaltase activity. Acta Paediatr Scand. 1971; 60:364. PMID: 5579866.
Aurrichio S. Glucoamylase Activity of Human Intestinal Mucosa. Rendic. R. Gasteroenterol 1971; 3:1-8.
Bayless T M, Christopher N L. Disaccharidase deficiency. Am. J. Clin. Nutr. 1969; 22:181-90. PMID:4885929.
Bird A R, Brown I L, Topping D L. Starches, resistant starches, the gut microflora and human health. Curr Intestin Microbiol 2000; 1:25-37. PMID: 11709851.
Borgstrom B. Dahlqvist A, Lundh G, Sjovall J. Studies of intestinal digestion and absorption in the human. J Clin Invest. 1957; 36:1521-36. PMID: 13475490.
Boudreau F, Zhu Y, Traber P G. Sucrase-isomaltase gene transcription requires the hepatocyte nuclear factor-1 (HNF-1) regulatory element and is regulated by the ratio of HNF-1 alpha to HNF-1 beta. J. Biol. Chem. 2001; 276:32122-8. PMID: 11395488.
Brown, H T, Heron, J. Some Observations on the Hydrolytic Ferments of the Pancreas and Small Intestine. Proc. Royal Soc. London. 1879-1880; 30: 393-407.
Calvin R T, Klish W J, Nichols B L. Disaccharide activities, jejunal morphology and carbohydrate tolerance in children with chronic diarrhea. J Pediatr. Gastroenterol. Nutr. 1985; 45:949-953. PMID: 4067784.
Caplan A, Walker L, Rasguin A. Validation of the pediatric Rome II criteria for functional gastrointestinal disorders using the questionnaire on pediatric gastrointestinal symptoms. J. Pediatr. Gastroenterol. Nutr. 2005; 41:305-16. PMID: 16131985.
Chitkara D K, Rawat D J, Talley N J. The epidemiology of childhood recurrent abdominal pain in Western countries: a systematic review. Am. J. Gastroenterol. 2005; 100:1868-75. PMID: 16086724.
Chitkara D K, Talley N J, Schleck C, Zinsmeister A R, Shah N D, Locke G R 3^{rd}. Recollection of childhood abdominal pain in adults with functional gastrointestinal disorders. Scand. J. Gastroenterol. 2009; 44:301-7. PMID: 19016078.
Dahlqvist A, Auricchio S, Semanza G, Prader A. Human intestinal disaccharidases and hereditary disaccharide intolerance. The hydrolysis of sucrose, isomaltose, palatinose (isomaltulose), and a 1,6-alpha-oligosaccharide (isomalto-oligosaccharide) preparation. J. Clin. Invest. 1963; 42:556-62. PMID: 14024642.
Escaffit F, Pare F, Gauthier R, Rivard N, Boudreau F, Beaulieu J F. Cdx2 modulates proliferation in normal human intestinal epithelial crypt cells. Biochem Biophys Res Commun. 2006; 342:66-72. PMID: 16480684.
Fernandez-Banares F, Esteve-Pardo M, deLeon R, Humbert P, Cabre E, Llovet J M, Gassull M A. Sugar malabsorption in functional bowel disease: clinical implications. Am J Gastroenterol 1993; 88:2044-50. PMID: 8249972.
Galand G. Brush border membrane sucrase-isomaltase, maltase-glucoamylase and trehalase in mammals. Comparative development, effects of glucocorticoids, molecular mechanisms, and phylogenetic implications. Comp Biochem Physiol B 1989; 94:1-11. PMID: 2513162.
Gilger M A, Gold B D. Pediatric endoscopy: new information from the PEDS-CORI project. Curr Gastroenterol Rep. 2005; 7:234-9. PMID: 15913484.
Goldstein R, Braverman D, Stankiewicz H. Carbohydrate malabsorption and the effect of dietary restriction on symptoms of irritable bowel syndrome and functional bowel complaints. Isr Med Assoc J 2000; 2:583-7. PMID: 10979349.
Gonçalvez A A, Badiale-Furlong E, de Souza-Soares L A, Siervs S T. Enzymatic determination of soluble and insoluble dietary fiber in rice and wheat bran. Arch Latinoam Nutr 1998; 48:334-8. PMID: 10347699.
Graham D Y, Klein P D, Evans D J Jr, Evans D G, Alpert L C, Opekun A R, Boutton T W. Campylobacter pylori detected noninvasively by the 13C-urea breath test. Lancet. 1987; 1(8543):1174-7. PMID: 2883491
Guzmán-Maldonado H, Paredes-LOpez 0. Amylolytic enzymes and products derived from starch: a review. Crit Rev Food Sci Nutr 1995; 35:373-403. PMID: 8573280.
Heymann H, Breitmeier Cs, Gunther S. Human small intestinal sucrase-isomaltase: different binding patterns for malto- and isomaltooligosaccharides. Biol Chem Hoppe Seyler. 1995; 376:249-53. PMID: 7626234.
Hyams J S. Irritable bowel syndrome, functional dyspepsia, and functional abdominal pain syndrome. Adolesc Med Clin. 2004; 15:1-15. PMID: 15272253.
Jamison R N, Gracely R H, Raymond S A, Levine J G, Marino B, Herrmann T J, Daly M, Fram D, Katz N P. Comparative study of electronic vs. paper VAS ratings: a randomized, crossover trial using healthy volunteers. Pain. 2002; 99:341-7. PMID: 12237213.
Jenkins D J, Jenkins A L, Wolever T M, Collier G R, Rao A V, Thompson L U. Starchy foods and fiber: reduced rate of digestion and improved carbohydrate metabolism. Scand J Gastroenterol 1987; 129:132-41. PMID: 2820027.
Jenkins D J, Taylor R H, Goff D V, Fielden H, Misiewicz J J, Sarson D L, Bloom S R, Alberti K G. Scope and specificity of acarbose in slowing carbohydrate absorption in man. Diabetes 1981; 30:951-4. PMID: 7028548.
Karnsakul W, Luginbuehl U, Hahn D, Sterchi E, Avery S, Sen P, Swallow D, Nichols B. Disaccharidase activities in dyspeptic children: biochemical and molecular investigations of maltase-glucoamylase activity. J Pediatr Gastroenterol Nutr 2002; 35:551-6. PMID: 12394383.
Keating J, Bjarnason I, Somadundaram S, MacPgerson A, Francis N, Price A B, Sharpstone D, Smithson J, Manzies I S, Qazzard B G. Intestinal absorptive capacity, intestinal permeability and jejunal histology in W V and their relation to diarrhea. Gut. 1995; 37:623-9.
Ladas S D, Frydas A, Papadopoulos A, Raptis S A. Effects of alpha-glucosidase inhibitors on mouth to caecum transit time in humans. Gut 1992: 1246-8. PMID: 1427379.
Lages L C A, Tannenbaum S R, Production of Glucose from Tapioca (casave starch) and forinha de mandicea (casava meal). J. Food Science 1978, 43:1012-1014.
Lane M M, Weider E M, Czyewski D I, Shulman R J. Pain symptoms and stooling patterns do not drive diagnostic costs for children with functional abdominal pain and irritable bowel syndrome in primary or tertiary care. Pediatrics 2009; 123:758-64. PMID: 19254999.
Lane S J, Heddle N M, Arnold E, Walker I. A review of randomized controlled trials comparing the effectiveness of hand held computers with paper methods for data collection. BMC Med Inform Decis Mak. 2006; 6:23. PMID 16735537.
Lebenthal E, Khin-Maung-U, Zheng B Y, Lu R B, Lerner A. Small intestinal glucoamylase deficiency and starch malabsorption: a newly recognized alpha-glucosidase deficiency in children. J Pediatr 1994; 124:541-6. PMID: 8151467.
Lee P C, Werlin S, Trost B, Struve M. Glucoamylase activity in infants and children: normal values and relationship to symptoms and histological findings. J Pediatr Gastroenterol 2004; 39:161-5. PMID: 15269621.
Li M, Hanford M J, Kim J W, Peeples T L. Amyloglucosidase enzymatic reactivity inside lipid vesicles. J Biol Eng 2007; 1:4. PMID: 18271982.
Lieberman D, Fennerty M B, Morris C D, Holub J, Eisen G, Sonnenberg A. Endoscopic evaluation of patients with dyspepsia: results from the national endoscopic data repository. Gastroenterol 2004; 127:1067-75. PMID: 15480985.
Lifschitz C H, Boutton 1 W, Carrazza F, Beyreiss K, Schmitz J, Ricour C, Shulman R, Nichols B L. A carbon-13 breath test to characterize glucose absorption and utilization in children. J Pediatr Gastroenterol Nutr 1989; 7:842-7.
Lomer M C, Parkes G C, Sanderson J D. Review article: lactose intolerance in clinical practice—myths and realities. Aliment Pharmacol Ther 2008; 27:93-103. PMID: 17956597.
Luceri C, Caderni G, Lodovici M, Spagnesi M Y, Monserrat C, Lancioni L, Dolara P. thinary excretion of sucrose and fructose as a predictor of sucrose intake in dietary intervention studies. Cancer Epidemiol Biomarkers Prey. 1996; 5:167-71.
Malaty H M, Abudayyeh S, O'Malley K J, Wilsey M J, Fraley K, Gilger M A, Hollier D, Graham D Y, Rabeneck L. Development of a multidimensional measure for recurrent abdominal pain in children: population-based studies in three settings. Pediatrics. 2005; 115:e210-5. PMID: 15687428.
Metha, D. I.; Blecker U. Chronic diarrhea in infancy and childhood. J. La. State Med. Soc., 1998, 150:419-429.
Moukarzel A A, Lesicka H, Ament M E. Irritable bowel syndrome and nonspecific diarrhea in infancy and childhood-relationship with juice carbohydrate malabsorption. Clin Pediatr (Phila) 2002; 41:145-50. PMID: 11999677.
Natarajan S, Sierks M R. Functional and structural roles of the highly conserved Trp120 loop region of glucoamylase from Aspergillus awamori. Biochemistry 1996; 35:3050-8. PMID: 8608145.
Nichols B L, Avery S, Sen P, Swallow D M, Hahn D, Sterchi E. The maltase-glucoamylase gene: Common ancestry to sucrase-isomaltase with complementary starch digestion activities. Proc Natl Acad Sci USA 2003; 100:1432-1437. PMID: 12547908.
Nichols B L, Avery S E, Karnsakul W, Jahoor F, Sen P, Swallow D M, Luginbuehl U, Hahn D, Sterchi E E. Congenital maltase-glucoamylase deficiency associated with lactase and sucrase deficiencies. J Pediatr Gastroenterol Nutr. 2002; 35:573-9. PMID: 12394387.
Nichols B L, Carrazza F, Nichols V N, Putman M, Johnston P, Rodrigues M, Quaroni A, Shiner M. Mosaic expression of brush-border enzymes in infants with chronic diarrhea and malnutrition. J Pediatr Gastroenterol Nutr. 1992; 14:371-9. PMID: 1355533.
Nichols B L, Eldering J, Avery S, Hahn D, Quaroni A, Sterchi E. Human small intestinal maltaseglucoamylase cDNA cloning. Homology to sucrase-isomaltase. J Biol Chem. 1998; 273:3076-81. PMID: 9446624.
Nichols B L, Quezada-Calvillo R, Robayo-Torres C C, Ao Z, Hamaker B R, Butte N F, Marini J, Jahoor F, Sterchi E E. Mucosal maltase-glucoamylase plays a crucial role in starch digestion and prandial glucose homeostasis of mice. J Nutr 2009; 139:684-90. PMID: 19193815.
Nichols, B L, Avery, S E, Butte, N F, Opekun, A R, Hahn, D, Sterchi, E E. Dietary Supplements of Yeast Amyloglucosidase Increase Proximal Starch Digestion in Maltaseglucoamylase (MGA) Knockout and Wild-type Mice. FASEB J. 2002; 16:A245.
Nichols, B L, Avery, S E, Karnsakul, W, Jahoor, F, Sen, P, Swallow, D M, Luginbuelil U, Hahn, D, Sterchi, E R Congenital Maltase-glucoamylase Deficiency Associated with Lactase and Sucrase Deficiencies: Pleiotropic gene replation? JPGN 2002; 35:573-579.
Nicklas T A, Myers L, Farris R P, Srinivasan S R, Berenson G S. Nutritional quality of a high carbohydrate diet as consumed by children: The Bogalusa Heart Study. J Nutr. 1996; 126:1382-8. PMID: 8618134.
Nordgaard 1, Mortensen P B. Digestive processes in the human colon. Nutrition 1995; 11:37-45. PMID: 7749242.
Quezada-Calvillo R, Robayo-Torres C, Ao Z. Luminal Substrate “Brake” on Mucosal Maltaseglucoamylase Activity Regulates Total Rate of Starch Digestion to Glucose. J Pediatr Gastroenterol Nutr. 2007; 45:32-43. PMID: 17592362.
Quezada-Calvillo R, Robayo-Torres C C, Opekun A R, Sen P, Ao Z, Hamaker B R, Quaroni A, Brayer G D, Wattler S, Nehls M C, Sterchi E E, Nichols B L. Contribution Of Mucosal Maltase-Glucoamylase Activities To Mouse Small Intestine Starch alpha-Glucogenesis. J Nutr 2007: 137, 1725-1733. PMID: 17585022.
Quezada-Calvillo R, Sim L, Ao Z, Hamaker B. R, Quaroni A, Brayer G D, Sterchi E E, Robayo-Torres C C, Rose D R, Nichols B L. Luminal Starch Substrate “Brake” on Maltase-Glucoamylase Activity Is Located within the Glucoamylase Subunit. The Journal of Nutrition 2008; 138:685-692. PMID: 18356321.
Rasquin A, Di Lorenzo C, Forbes D, Guiraldes E, Hyams J S, Staiano A, Walker L S. Childhood functional gastrointestinal disorders: child/adolescent. Gastroenterol 2006; 130:1527-1537. PMID 16678566.
Rasquin-Weber A, Hyman P E, Cucchiara S, Fleisher D R, Hyams J S, Milla P J, Staiano A. Childhood functional gastrointestinal disorders. Gut 1999; 45 Suppl 2:1160-8. PMID: 10457047.
Robayo-Torres C C, Opekun A R, Quezada-Calvillo R, Villa X, Smith E O, Navarrete M, Baker S S, Nichols B L. ¹³C-breath tests for sucrose digestion in congenital sucrase isomaltase-deficient and sacrosidase supplemented patients. J Pediatr Gastroenterol Nutr. 2009; 48:412-8. PMID: 19330928.
Robayo-Torres C C, Quezada-Calvillo R, Nichols B L. Disaccharide digestion: clinical and molecular aspects. Clin Gastroenterol Hepatol. 2006; 4:276-87. PMID: 16527688.
Russo M W, Wei J T, Thiny M T, Gangarosa L M, Brown A, Ringel Y, Shaheen N J, Sandler R S. Digestive and liver diseases statistics, 2004. Gastroenterol. 2004; 126:1448-53. PMID: 15131804.
Sauer J, Sigurskjold B W, Christensen U, Frandsen T P, Mirgorodskaya E, Harrison M, Roepstorff P, Svensson B. Glucoamylase: structure/function relationships, and protein engineering. Biochim Biophys Acta 2000; 1543:275-293. PMID: 11150611.
Shulman R J, Lifschitz C H, Langston C, Gopalakrishna G S, Nichols B L. Human milk and the rate of small intestinal mucosal recovery in protracted diarrhea. J Pediatr. 1989; 114:218-24. PMID: 2492597.
Silberg D G, Swain G P, Suh E R, Traber P G. Cdx1 and cdx2 expression during intestinal development. Gastroenterology. 2000; 119:961-71. PMID: 11040183.
Sim L, Quezada-Calvillo R, Sterchi E. E., Nichols B L, Rose D R. Human Intestinal Maltase-Glucoamylase: Crystal Structure of the N-Terminal Catalytic Subunit and Basis of Inhibition and Substrate Specificity. Journal of Molecular Biology 2007; 3:782-792. PMID: 18036614.
Simadibrata M, Wanders R J, Jan G, Tytgat G N, Lesmana L A, Daldiyono Ariawan I., Examination of small bowel enzymes in chronic diarrhea. Journal of Gastroenterology and Hepatology 2003; 18:53-56.
Slavin J. Why whole grains are protective: biological mechanisms. Proc Nutr Soc 2003; 62:129-34. PMID: 12740067.
Stone A A, Shiffman S, Schwartz J E, Broderick J E, Hufford M R. Patient compliance with paper and electronic diaries. Control Clin Trials 2003; 24:182-99. PMID 12689739.
Tasevska N, Runswick S A, Welch A A, McTaggart A, Bingham S A. Urinary sugars biomarker relates better to extrinsic than to intrinsic sugars intake in a metabolic study with volunteers consuming their normal diet. Eur J Clin Nutr. 2009; 63:653-9.
Taylor C, Hodgson K, Sharpstone D, Sigthorsson G, Coutts M, Sherwood R, Menzies I, Gazzard B, Bjarnason I. The prevalence and severity of intestinal disaccharidase deficiency in human immunodeficiency virus-infected subjects. Scand J Gastroenterol. 2000; 35:599-606. PMID: 10912659.
Treem W R, McAdams L, Stanford L, Kastoff G, Justinich C, Hyams J. Sacrosidase therapy for congenital sucrase-isomaltase deficiency. J Pediatr Gastroenterol Nutr1999; 28:137-42. PMID: 9932843.
Van Munster I P, Tangerman A, Nagengast F M. Effects of starch on colonic fermentation, bile acid metabolism and mucosal proliferation. Dig Dis Sci. 1994; 39:834-42. PMID: 8149850.
Walker L S, Guite J W, Duke M, Barnard J A, Greene J W. Recurrent abdominal pain: a potential precursor of irritable bowel syndrome in adolescents and young adults. J Pediatr. 1998; 132:1010-15. PMID: 9627595.
Walker L S, Lipani T A, Greene J W, Caines K, Stutts J, Polk D B, Caplan A, Rasguin-Weber A. Recurrent abdominal pain: symptom subtypes based on the Rome 11 Criteria for pediatric functional gastrointestinal disorders. J Pediatr Gastroenterol Nutr. 2004; 38:187-91. PMID: 14734882.
Wright E M. Sugar Absorption. In Johnson L R, ed. Physiology of the gastrointestinal tract. Vol. II, 4th Ed. New York: Academic Press, 2006; 1653-1665.
Youssef N N, Murphy T G, Langseder A L, Rosh J R. Quality of life for children with functional abdominal pain: a comparison study of patients' and parents' perceptions. Pediatrics 2006; 117:54-9. PMID: 16396860.

1. A composition having the general formula: 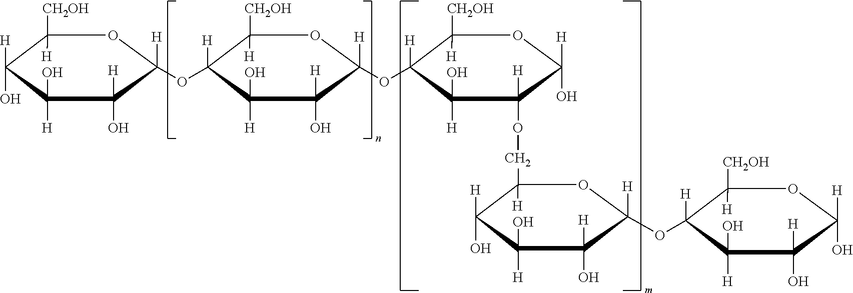
wherein the composition is uniformly enriched with ¹³C or ¹⁴C;
wherein the composition has between 5 and 60 glucose units;
wherein n is between 3 and 58;
wherein m is between 0 and 10; and
wherein the sum of m and n is between 3 and 58.

2. The composition of claim 1, wherein when m is 1 or more the ratio of m and n is between 1 to 58 and 1 to 3.

3. The composition of claim 1, wherein n is between 11 and 30, between 15 and 26, between 19 and 22, or n is about 20.

4. (canceled)

5. (canceled)

6. (canceled)

7. The composition of claim 1, wherein m is between 1 and 5, between 1 and 3, m is 2, or m is 1.

8. (canceled)

9. (canceled)

10. (canceled)

11. The composition of claim 1, wherein the sum of m and n is about 20, m is 1 and n is 19.

12. A solution comprising,
a ¹³C-α-limit dextrin, or a ¹⁴C-α-limit dextrin having the general formula: 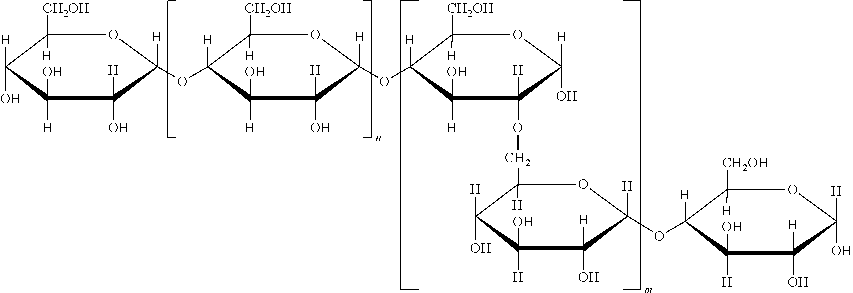
wherein n is between 3 and 58;
wherein m is between 0 and 10; and
wherein the sum of m and n is between 3 and 58.

13. The solution of claim 12, wherein when m is 1 or more the ratio of m and n is between 1 to 58 and 1 to 3.

14. The solution of claim 12, wherein n is between 11 and 30, between 15 and 26, between 19 and 22, or n is about 20.

15. (canceled)

16. (canceled)

17. (canceled)

18. The solution of claim 12, wherein m is between 1 and 5, between 1 and 3, m is 2, or m is 1.

19. (canceled)

20. (canceled)

21. (canceled)

22. The solution of claim 12, wherein the sum of m and n is about 20, m is 1 and n is 19.

23. A method for detecting intestinal enzyme deficiency in a subject comprising the steps of:
collecting a breath sample for measurement of baseline (natural enrichment) ¹³C or ¹⁴C labeled carbon dioxide for subsequent, timed comparisons (change in enrichments);
administering to a subject a uniformly ¹³C or ¹⁴C labeled α-limit dextrin substrate (or other target substrate) with concomitant excipient load of unlabeled dextrins (or unlabeled other target substrate), wherein the uniformly ¹³C or ¹⁴C labeled α-limit dextrin substrate (or other target substrate) is metabolized to produce ¹³CO₂ or ¹⁴CO₂,
measuring the timed change in ¹³CO₂ or ¹⁴CO₂ enrichment resulting from the metabolized uniformly ¹³C or ¹⁴C labeled α-limit dextrin substrate (or other target substrate) by collecting breath samples from the subject;
administering to the subject a uniformly ¹³C or ¹⁴C labeled glucose substrate, wherein the uniformly ¹³C or ¹⁴C labeled glucose substrate is metabolized to produce ¹³CO₂ or ¹⁴CO₂,
measuring the timed change of ¹³CO₂ or ¹⁴CO₂ enrichment resulting from the metabolized uniformly ¹³C or ¹⁴C labeled glucose substrate by collecting breath samples from the subject; and
comparing the measured ¹³CO₂ or ¹⁴CO₂ resulting from the metabolized uniformly ¹³C or ¹⁴C labeled α-limit dextrin substrate (or other target substrate) with the measured ¹³CO₂ or ¹⁴CO₂ resulting from the metabolized uniformly ¹³C or ¹⁴C labeled glucose after a proportional adjustment for superdosing of uniformly ¹³C or ¹⁴C labeled glucose substrate and expressing the resultant values as a ratio.

24. The method of claim 23, wherein the intestinal enzyme deficiency results from a genetic mutation.

25. The method of claim 24, wherein the genetic mutation is located on the sucrase-isomaltase gene or is located on the maltase-glucoamylase gene.

26. (canceled)

27. The method of claim 23, wherein the intestinal enzyme deficiency is an acquired intestinal enzyme deficiency.

28. The method of claim 23, wherein the substrate has the general formula: 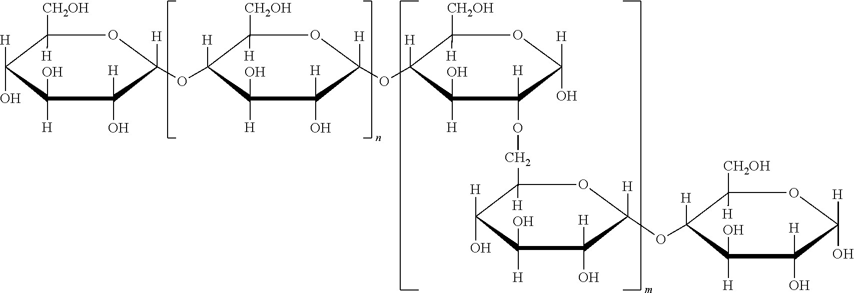
wherein n is between 3 and 58;
wherein m is between 0 and 10; and
wherein the sum of m and n is between 3 and 58.

29. The method of claim 28, wherein when m is 1 or more the ratio of m and n is between 1 to 58 and 1 to 3.

30. The method of claim 28, wherein n is between 11 and 30, between 15 and 26, between 19 and 22, or n is about 20.

31. (canceled)

32. (canceled)

33. (canceled)

34. The method of claim 28, wherein m is between 1 and 5, between 1 and 3, m is 2, or m is 1.

35. (canceled)

36. (canceled)

37. (canceled)

38. The method of claim 28, wherein the sum of m and n is about 20, m is 1 and n is 19.

39. The method of claim 23, wherein the other target substrate is:

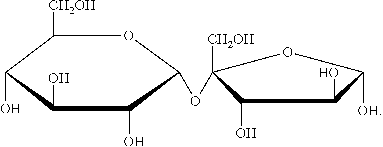

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method for monitoring a subject having been diagnosed with intestinal enzyme deficiency comprising the steps of:
administering to the subject a uniformly ¹³C or ¹⁴C labeled substrate, wherein the uniformly ¹³C or ¹⁴C labeled substrate is metabolized to produce ¹³CO₂ or ¹⁴CO₂;
measuring the ¹³CO₂ or ¹⁴CO₂ resulting from the metabolized uniformly ¹³C or ¹⁴C labeled substrate by collecting breath samples from the subject;
administering to a subject a uniformly ¹³C or ¹⁴C labeled glucose, wherein the uniformly ¹³C or ¹⁴C glucose is metabolized to produce ¹³CO₂ or ¹⁴CO₂;
measuring the ¹³CO₂ or ¹⁴CO₂ resulting from the metabolized uniformly ¹³C or ¹⁴C labeled glucose by collecting breath samples from the subject; and
comparing the measured ¹³CO₂ or ¹⁴CO₂ resulting from the metabolized uniformly ¹³C or ¹⁴C labeled substrate with the measured ¹³CO₂ or ¹⁴CO₂ resulting from the metabolized uniformly ¹³C or ¹⁴C labeled glucose after a proportional adjustment for superdosing of labeled glucose.

52. The method of claim 51, wherein the intestinal enzyme deficiency results from a genetic mutation.

53. The method of claim 52, wherein the genetic mutation is located on the sucrase-isomaltase gene or is located on the maltase-glucoamylase gene.

54. (canceled)

55. The method of claim 51, wherein the intestinal enzyme deficiency is an acquired intestinal enzyme deficiency.

56. The method of claim 51, wherein the substrate has the general formula: 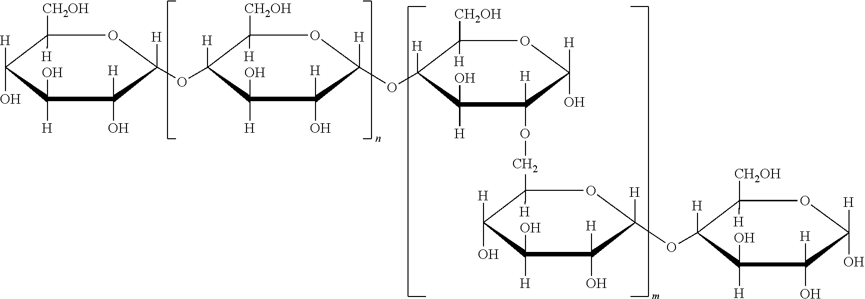
wherein n is between 3 and 58;
wherein m is between 0 and 10; and
wherein the sum of m and n is between 3 and 58.

57. The method of claim 56, wherein when m is 1 or more the ratio of m and n is between 1 to 58 and 1 to 3.

58. The method of claim 56, wherein n is between 11 and 30, between 15 and 26, between 19 and 22, or n is about 20.

59. (canceled)

60. (canceled)

61. (canceled)

62. The method of claim 56, wherein m is between 1 and 5, between 1 and 3, m is 2, or m is 1.

63. (canceled)

64. (canceled)

65. (canceled)

66. The method of claim 56, wherein the sum of m and n is about 20, m is 1 and n is 19.

67. The method of claim 51, wherein the substrate has the formula:

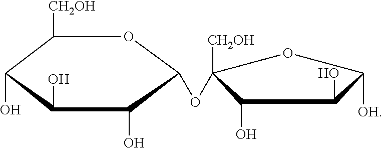

68. A method for treating a subject having been diagnosed with intestinal enzyme deficiency comprising the steps of:
administering to the subject a uniformly ¹³C or ¹⁴C labeled substrate, wherein the uniformly ¹³C or ¹⁴C labeled substrate is metabolized to produce ¹³CO₂ or ¹⁴CO₂;
measuring the ¹³CO₂ or ¹⁴CO₂ resulting from the metabolized uniformly ¹³C or ¹⁴C labeled substrate by collecting breath samples from the subject;
administering to a subject a uniformly ¹³C or ¹⁴C labeled glucose, wherein the uniformly ¹³C or ¹⁴C glucose is metabolized to produce ¹³CO₂ or ¹⁴CO₂;
measuring the ¹³CO₂ or ¹⁴CO₂ resulting from the metabolized uniformly ¹³C or ¹⁴C labeled glucose by collecting breath samples from the subject; and
comparing the measured ¹³CO₂ or ¹⁴CO₂ resulting from the metabolized uniformly ¹³C or ¹⁴C labeled substrate with the measured ¹³CO₂ or ¹⁴CO₂ resulting from the metabolized uniformly ¹³C or ¹⁴C labeled glucose after a proportional adjustment for superdosing of labeled glucose.

69. The method of claim 66, further comprising the step of administering to the subject amyloglucosidase.

70. The method of claim 68, wherein the substrate has the general formula: 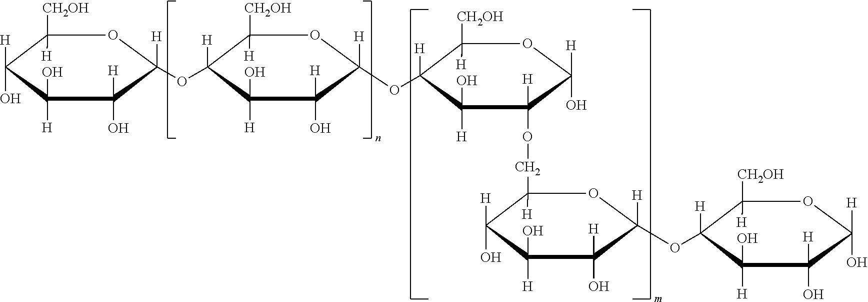
wherein n is between 3 and 58;
wherein m is between 0 and 10; and
wherein the sum of m and n is between 3 and 58.

71. The method of claim 70, wherein when m is 1 or more the ratio of m and n is between 1 to 58 and 1 to 3.

72. The method of claim 70, wherein n is between 11 and 30, between 15 and 26, between 19 and 22, or n is about 20.

73. (canceled)

74. (canceled)

75. (canceled)

76. The method of claim 70, wherein m is between 1 and 5, between 1 and 3, m is 2, or m is 1.

77. (canceled)

78. (canceled)

79. (canceled)

80. The method of claim 70, wherein the sum of m and n is about 20, m is 1 and n is 19.

81. The method of claim 68, wherein the intestinal enzyme deficiency results from a genetic mutation.

82. The method of claim 81, wherein the genetic mutation is located on the sucrase-isomaltase gene or is located on the maltase-glucoamylase gene.

83. (canceled)

84. The method of claim 68, wherein the intestinal enzyme deficiency is an acquired intestinal enzyme deficiency.
